(19) United States
(12) Patent Application Publication
(10) Pub. No.: US 2022/0105183 A1
(43) Pub. Date: Apr. 7, 2022

(54) MICRO-NANO STRUCTURE FORMED BY SELF-ASSEMBLING ORGANIC SMALL MOLECULE COMPOUND AND USE THEREOF

(71) Applicant: Qingdao University of Science and Technology, Qingdao, Shandong (CN)

(72) Inventors: Xianfeng ZHOU, Qingdao, Shandong (CN); Zhibo LI, Qingdao, Shandong (CN); Xueluer MU, Qingdao, Shandong (CN)

(21) Appl. No.: 17/296,166

(22) PCT Filed: Nov. 14, 2019
(86) PCT No.: PCT/CN2019/118529

§ 371 Date: Dec. 13, 2021

(30) Foreign Application Priority Data
Nov. 22, 2018 (CN) 201811399004.6
Nov. 22, 2018 (CN) 201811399021.X
Nov. 22, 2018 (CN) 201811400036.3

(51) Int. Cl. A61K 41/00; A61K 49/22; A61P 35/00; C07D 209/10; C07D 405/08

(52) U.S. Cl. CPC A61K 41/0052; A61K 49/22; C07D 405/08; C07D 209/10; A61P 35/00

(57) ABSTRACT

A micro-nano structure formed by self-assembling a compound represented by formula (I), an isomer thereof, a pharmaceutically acceptable salt, a hydrate or a solvate in an aqueous solution, a preparation method for the micro-nano structure, and use thereof are described. The micro-nano structure has the advantages of having high photothermal conversion efficiency, good photothermal stability, good photothermal effect and photodynamic effect, being easily degraded, and having high safety, and can be passively targeted to tumor sites, having a broad prospect in the diagnosis and treatment of cancers and skin diseases.

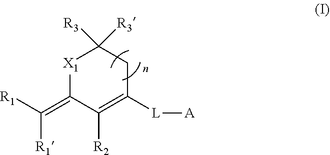

### TECHNICAL FIELD

The invention relates to a new type of micro-nano structure formed by self-assembly of a small organic fluorescent compound and its application, in particular to a type of fluorescence compound that emits heat while emits light under laser irradiation, and the temperature rises to kill tumor cells to achieve a healing effect and the micro-nano structure formed by the self-assembly of the compound, belong to the field of chemical and pharmaceutical.

### BACKGROUND ART

In recent years, the incidence of cancer has been raised, and there is a major threat to people's life and health. Existing treatment techniques such as surgical treatment, chemotherapy methods have certain limitations. Therefore, laser photothermal treatment gradually enters into people's vision, which is a cancer treatment method with clinical application prospects, with non-invasive/mini-invasive advantages, greatly reduces the pain of patients. The method is to irradiate tumor tissue with a beam of near-infrared light, the fluorescent compound will generate heat while emitting light, and the temperature thereof will rise to kill cancer cells to achieve a therapeutic effect. This method has few side reactions and high selectivity.

Since conventional organic small molecular fluorescent compounds (FIG. 1) typically have the disadvantage of poor photothermal stability in photothermal therapy, many researchers have studied inorganic nanomaterials as photothermal agents for photothermal therapy of cancer in recent years. Although inorganic nanomaterials can have high light-to-heat conversion efficiency, their clinical development and applications are limited because they are generally not easily degraded in vivo and have potential toxicity problems.

In addition, in the prior art, it has been reported that the macromolecular groups such as PEG are attached to the organic small molecule fluorescent compound to increase its photothermal conversion efficiency and photothermal stability, but the fluorescent compound is still facing metabolic difficulties and potential toxicity problem.

Therefore, it is of great significance to study small organic fluorescent compounds with excellent photothermal stability for laser photothermal treatment of cancer.

### SUMMARY OF THE INVENTION

The present invention is directed to the problems in the prior art, and provides a type of new type of uncharged organic small molecular fluorescent compound and its application in phototherapy. Such compounds can self-assemble into micro-nano structures in water, and have the advantages of high light-to-heat conversion efficiency, excellent photothermal stability, easy degradation and high safety which can be used for phototherapy of cancer in vivo.

Accordingly, in one aspect, the present invention provides a micro-nano structure formed by self-assembly of a compound having the structure represented by formula (I), an isomer, a pharmaceutically acceptable salt, a hydrate or a solvate in an aqueous solution,

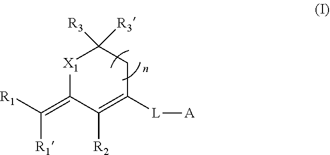

in formula (I):

A is a substituted or unsubstituted heterocyclyl, preferably the heterocyclyl is uncharged, more preferably the heterocyclyl contains one or more heteroatoms selected from the group consisting of N, O and S;

L is a substituted or unsubstituted conjugated carbon chain, preferably, the conjugated carbon chain contains 2-5 double bonds, more preferably, the number of double bonds in the conjugated carbon chain is 2, 3, 4 or 5;

X₁ is O, N or —CR₄R₄′—, preferably X₁ is O;

n is 0 or 1, preferably n is 0;

R₁, R₁′ and R₂ are each independently selected from atoms and groups with electron withdrawing ability, preferably R₁, R₁′ and R₂ are each independently selected from —CN, —CF₃, —F, —SO₂CF₃, —NO₂, —COOEt, —SO₂ph,

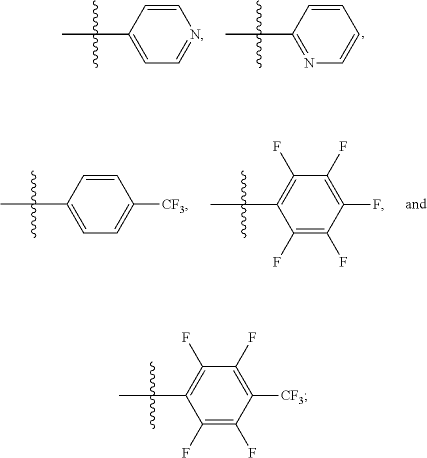 [SMILES: *c1ccncc1 ; *c1ccccn1 ; *c1ccc(C(F)(F)F)cc1 ; *c1c(F)c(F)c(F)c(F)c1F ; *c1c(F)c(F)c(C(F)(F)F)c(F)c1F]

more preferably, both R₁ and R₁′ are —CN; R₂ is —CN or

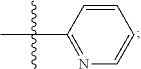 [SMILES: *c1ccccn1]

R₃ and R₃′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxyl, substituted or unsubstituted amido, substituted or unsubstituted ester group and substituted or unsubstituted amino; preferably, R₃ and R₃′ are each independently selected from H, —(CH₂)_{q}CH₃, —(CH₂)_{q}CF₃, —(CH₂)_{q}CH═CH₂, —(CH₂)_{q}C≡CH, —(CH₂)_{q}OH, —(CH₂)_{q}COOH, —(CH₂)_{q}NH₂, —(CH₂)_{q}CHO, —(CH₂)_{q}CO(CH₂)_{q}, CH₃, —(CH₂)_{q}O(CH₂)_{q′}CH₃,

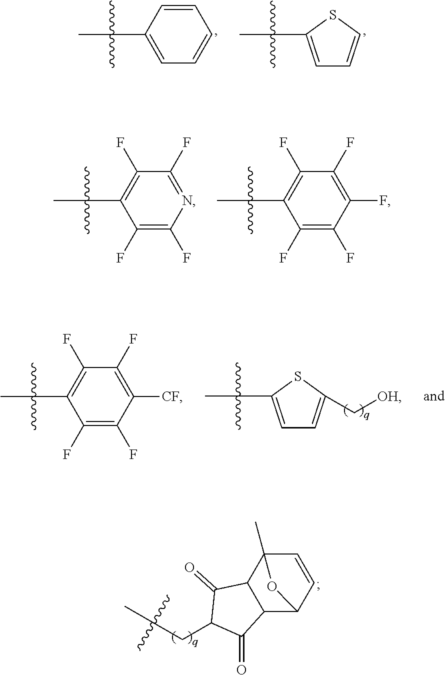

wherein q and q′ are each independently an integer selected from 0-12;

R₄ and R₄′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino;

when the group is substituted, the substituent is single or multiple.

The micro-nano structure formed by the self-assembly of the compound of formula (I) in an aqueous solution has the advantages of high light-to-heat conversion efficiency, excellent photothermal stability, easy degradation and high safety.

In some preferred embodiments, A is selected from substituted or non-substituted pyrrole or hydrogenated pyrrole ring, substituted or unsubstituted furan or hydrogenated furan ring, substituted or unsubstituted thiophene or hydrogenated thiophene ring, substituted or unsubstituted pyrazole or hydrogenated pyrazole ring, substituted or unsubstituted imidazole or hydrogenated imidazole ring, substituted or unsubstituted oxazole or hydrogenated oxazole ring, substituted or unsubstituted isoxazole or hydrogenated isoxazole ring, substituted or unsubstituted thiazole or hydrogenated thiazole ring, substituted or unsubstituted indole or hydrogenated indole ring, substituted or unsubstituted benzofuran or hydrogenated benzofuran ring, substituted or unsubstituted benzimidazole or hydrogenated benzimidazole ring, substituted or unsubstituted carbazole or hydrogenated carbazole ring, substituted or unsubstituted pyridine or hydrogenated pyridine ring, substituted or unsubstituted pyran or hydrogenated pyran ring, substituted or unsubstituted thiopyran or hydrogenated thiopyran ring, substituted or unsubstituted benzopyrazole or hydrogenated benzopyrazole ring, substituted or unsubstituted pyridazine or hydrogenated pyridazine ring, substituted or unsubstituted pyrimidine or hydrogenated pyrimidine ring, substituted or unsubstituted pyrazine or hydrogenated pyrazine ring, substituted or unsubstituted piperidine ring, substituted or unsubstituted morpholine ring, substituted or unsubstituted thiomorpholine ring and substituted or unsubstituted triazole ring;

preferably, A

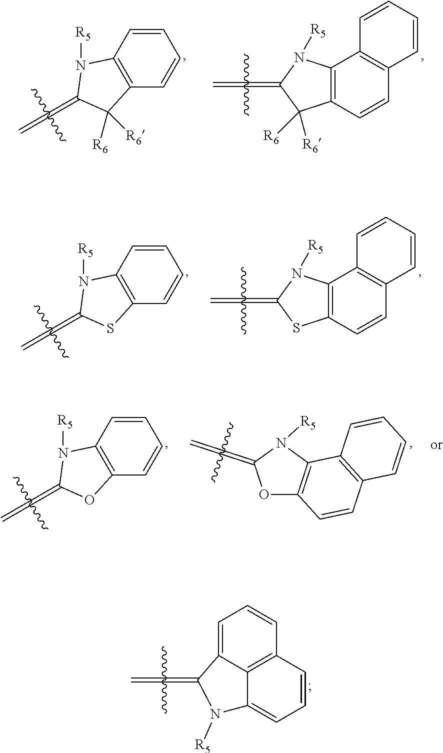

wherein R₅, R₆, R₆′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

In other preferred embodiments, L is

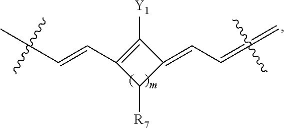

wherein

Y₁ is a halogen atom, a substituted or non-substituted amino or hydrocarbyloxy;

m is an integer of 0-5, preferably, m is 3;

each R₇ is independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

In a particularly preferred embodiment, m is 3, Y₁ is Cl, Br, —NR₈R₈′ or —OR₈; and, R₇ is H, —CH₃,

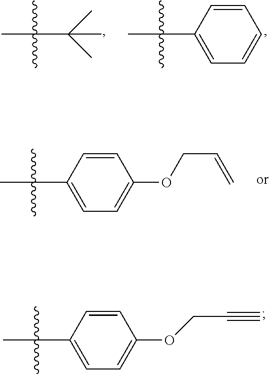

R₈ and R₈′ are each independently selected from H, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

The present invention further provides a micro-nano structure formed by self-assembly of a compound having the structure represented by formula (II), the isomer, pharmaceutically acceptable salt, hydrate or solvate thereof in an aqueous solution,

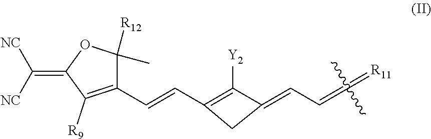

in the formula (II),

Y₂ is Cl, Br,

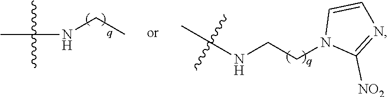

wherein q and q′ are each independently an integer selected from 0-12;

R₉ is —CN or

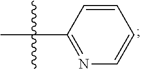 [SMILES: *c1ccccn1]

R₁₀ is (CH₂)ₘ—,

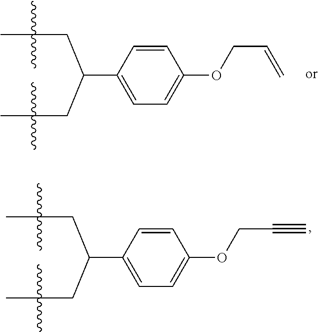 [SMILES: *CC(C*)c1ccc(OCC=C)cc1 ; *CC(C*)c1ccc(OCC#C)cc1]

m is an integer of 0-5, preferably R₁₀ is —CH₂—, —(CH₂)₂—, —(CH₂)₃— and —(CH₂)₄—, more preferably, R₁₀ is —(CH₂)₃—;

R₁₁ is

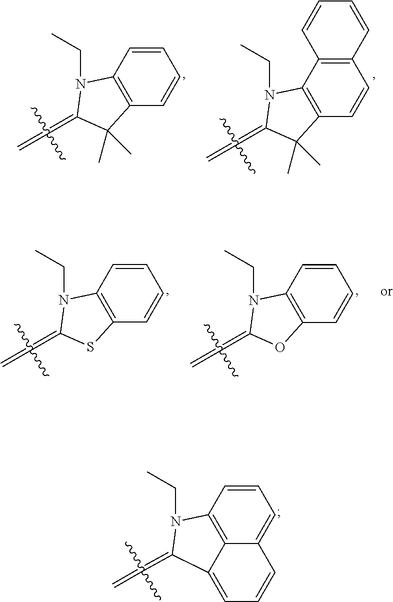 [SMILES: *=C1N(CC)c2ccccc2C1(C)C ; *=C1N(CC)c2c(ccc3ccccc23)C1(C)C ; *=C1Sc2ccccc2N1CC ; *=C1Oc2ccccc2N1CC ; *=c1c2cccc3cccc(c32)n1CC]

R₁₂ is

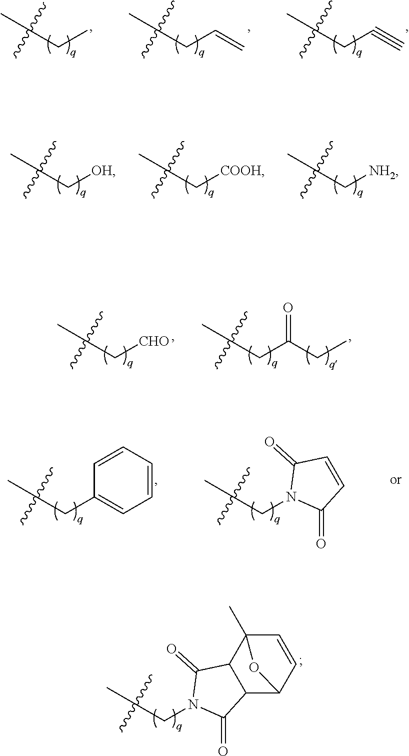

q and q′ are each independently an integer selected from 0-12;

more preferably, the micro-nano structure is formed by self-assembly of Compound II-1, II-2, II-3, II-4, II-5, II-6, II-7, II-8, II-9, II-10, II-11, II-12, II-13, II-14, II-15, II-16 or II-17 in aqueous solution.

Because the tumor (especially solid tumor) tissue is rich in blood vessels and lacks the lymphatic reflux system, the micro-nano structure described in the present invention can cause passive high permeability and retention at the tumor location. The high-permeability effect and retention effect of this micro-nano structure in solid tumor tissue is called the EPR effect (enhanced permeability and retention effect). This ability to passively target tumors makes these small molecule compounds that can be assembled by supramolecules to form micro-nano structures. They have obvious advantages over other reported small-molecule photothermal conversion reagents.

In a preferred embodiment of the invention, the particle size of the micro-nano structure is 1 nm-500 nm, preferably 10 nm-200 nm. More preferably, it is 30 nm-150 nm.

In another preferred embodiment of the present invention, the micro-nano structure is a nano-sheet structure formed by self-assembly of the compound having the structure represented by formula (I) or formula (II), the isomer, pharmaceutically acceptable salt, hydrate and solvate thereof in an aqueous solution.

Another aspect of the present invention also provides a method for preparing the micro-nano structure comprising the steps of.

1) dissolving the compound of formula (I) or formula (II), the isomer, pharmaceutically acceptable salt, hydrate or solvate thereof with organic solvent; preferably, the organic solvent is one or more of alkanes, olefins, aromatics, alcohols, ketones, aldehydes, carboxylic acids, esters or ethers; more preferably, the organic solvent is one or more of dimethyl sulfoxide, N,N-dimethylformamide, methanol, ethanol, ethylene glycol, n-propanol, isopropanol, propylene glycol, glycerol, n-butanol, isobutanol, butanediol or polyethylene glycol, acetone, dichloromethane or acetonitrile; in one preferred example, the organic solvent is ethanol;

2) adding the solution obtained in step 1) to the aqueous solution, preferably, in the system formed in step 2), the final concentration of the compound is 1 nM-1M; more preferably 10 nM-1 mM; more preferably 100 nM-500 μM; most preferably 0.46 μM-300 μM;

3) self-assembling the compound to form the micro-nano structure in the aqueous solution.

The above preparation method is simple, convenient, and suitable for large-scale production.

Another aspect of the invention also provides a pharmaceutical composition comprising:
1) a therapeutically effective dose of the micro-nano structure, and
2) pharmaceutically acceptable carrier; preferably, the pharmaceutically acceptable carrier comprises a diluent, disintegrant, excipient, adhesive, stabilizer, or combinations thereof.

The pharmaceutical composition preferably can be made into an injection comprising a therapeutically effective dose of the micro-nano structure and an injection solvent or additional agent or a combination thereof; preferably, the injection solvent is one, two or more mixed solvents of water for injection, ethanol, propylene glycol, glycerol and polyethylene glycol.

In some preferred embodiments, the micro-nano structure is a nano-sheet structure, and the pharmaceutical composition further includes an active agent encapsulated in the micro-nano structure, preferably a therapeutic agent or a diagnostic agent, more preferably a chemotherapeutic agent or radiotherapy agent, such as small molecule chemotherapy drugs, targeted therapy drugs, chemotherapy drugs, antibody drugs, etc. More preferably, the micro-nano structure further includes a targeting molecule, preferably an antibody, peptide, aptamer, or folic acid and the like.

In some preferred embodiments, the pharmaceutical composition is an injection.

Another aspect of the present invention also provides a use of the micro-nano structure or the pharmaceutical composition in the preparation of phototherapy drugs. Preferably, the phototherapy drugs are photothermal therapeutic drugs, photodynamic therapeutic drugs or photoacoustic therapeutic drugs.

The present invention also provides a use of the micro-nano structure or the pharmaceutical composition as a photosensitizer. Preferably, the photosensitizer is used to prepare photothermal therapeutic drugs, photodynamic therapeutic drugs or photoacoustic therapeutic drugs.

The present invention also provides a use of the micro-nano structure or the pharmaceutical composition in the preparation of drugs for diagnosis and/or treatment of cancer. Preferably, the cancer is esophageal cancer, non-small cell lung cancer, biliary cancer, head and neck cancer, Barrett esophagus, bladder cancer, colorectal cancer, pancreatic cancer, ovarian cancer, prostate cancer, brain tumor, breast cancer or skin cancer; the skin cancer includes melanoma.

The invention also provides a use of the micro-nano structure or the pharmaceutical composition in the preparation of a medicament for the treatment of skin diseases. Preferably, the skin diseases are actinic keratosis, basal cell carcinoma, and skin T-cell lymphoma, Bowen's disease, squamous cell carcinoma, intraepithelial neoplasia of the vulva and anus, or Paget's disease.

Another aspect of the present invention also provides a method for performing phototherapy on a target area of a subject, comprising:

1) providing the micro-nano structure;

2) administering the micro-nano structure to the subject;

3) waiting for the micro-nano structure to be enriched in the target area;

3) irradiating the target area of the subject with light in the excitation wavelength band of the micro-nano structure, preferably, 808 nm light is used to irradiate.

Another aspect of the invention provides a compound having a structure shown in formula (III), an isomer, pharmaceutically acceptable salt, hydrate or solvate thereof,

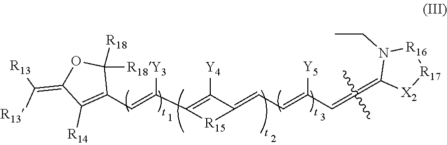

in the formula (III),

X₂ is selected from O, S or —CR₂₀R₂₀′—;

Y₃, Y₄ and Y₅ are each independently selected from H, hydroxyl, halogen atom, substituted or non-substituted amino and hydrocarbyloxy;

t₁, t₂ and t₃ are each independently an integer selected from 0-5, preferably both t₁ and t₂ are 1, and t₃ is 0;

R₁₃, R₁₃′ and R₁₄ are each independently selected from —CN, —CF₃, F, —SO₂CF₃, —NO₂, —COOEt, —SO₂ph,

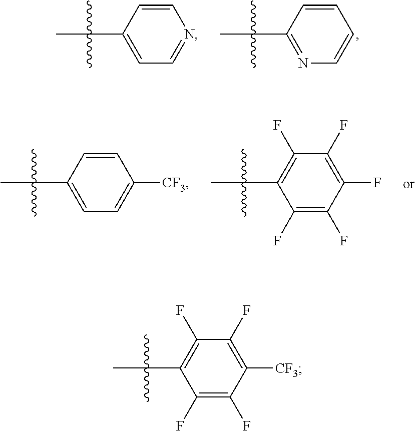 [SMILES: *c1ccncc1 ; *c1ccccn1 ; *c1ccc(C(F)(F)F)cc1 ; *c1c(F)c(F)c(F)c(F)c1F ; *c1c(F)c(F)c(C(F)(F)F)c(F)c1F]

preferably, both R₁₃ and R₁₃′ are —CN, R₁₄ is —CN or

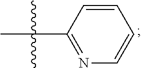 [SMILES: *c1ccccn1]

R₁₅ is —(CH₂)ₘ—,

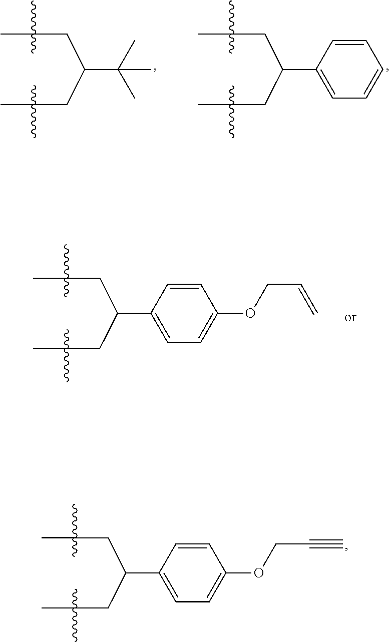

m is an integer of 0-5, preferably m is 3;

R₁₆ and R₁₇ together form one of the following connections:

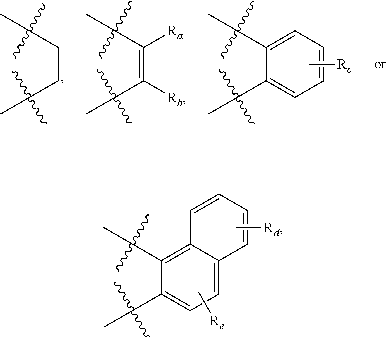

or R₁₆, R₁₇ and X₂ together form a connection

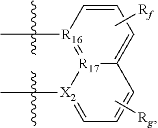

wherein Rₐ, R_{b}, R_{c}, R_{d}, Rₑ, R_{f} and R_{g} are each independently selected from H, halogen atom, substituted or non-substituted hydrocarbyl, substituted or non-substituted carboxyl, substituted or non-substituted hydroxyl and substituted or non-substituted amino;

R₁₈, R₁₈′, R₂₀ and R₂₀′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxyl, substituted or unsubstituted amido, substituted or unsubstituted ester group and substituted or unsubstituted amino; preferably, R₁₈ and R₁₈′ are each independently selected from H, —(CH₂)_{q}CH₃, —(CH₂)_{q}CF₃, —(CH₂)_{q}CHCH₂, —(CH₂)_{q}CCH, —(CH₂)_{q}OH, —(CH₂)_{q}COOH, —(CH₂)_{q}NH₂, —(CH₂)_{q}CHO, —(CH₂)_{q}CO(CH₂)_{q′}CH₃, —(CH₂)_{q}O(CH₂)_{r′}CH₃,

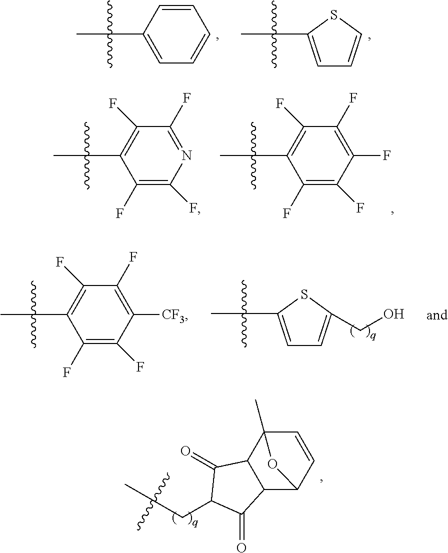

wherein q and q′ are each independently an integer selected from 0-12;

when the group is substituted, the substituent is single or multiple.

In a preferred embodiment,

both Y₃ and Y₅ are H;

Y₄ is Cl, Br or —NR₂₁R₂₁″—;

both t₁ and t₂ are 1, t₃ is 0;

R₂₁ and R₂₁′ are each independently selected from H, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

More preferably, the compound of formula (III) is Compound II-1, II-2, II-3, II-4, II-5, II-6, II-7, II-8, II-9, II-10, II-11, II-12, II-13, II-14, II-15, II-16 or II-17.

Another aspect of the invention provides a pharmaceutical composition comprising:
1) a therapeutically effective dose of the compound represented by formula (III), the isomer, pharmaceutically acceptable salt, hydrate or solvate thereof, and
2) pharmaceutically acceptable carrier; preferably, the pharmaceutically acceptable carrier comprises diluent, disintegrant, excipient, adhesive, stabilizer, or combinations thereof.

Another aspect of the present invention provides a use of a compound having the structure represented by formula (IV), the isomer, pharmaceutically acceptable salt, hydrate or solvate thereof in the preparation of a phototherapy drug,

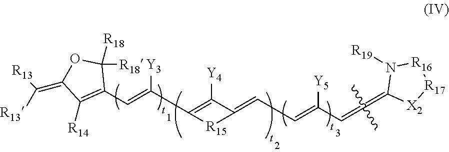

in formula (IV):

X₂ is selected from O, S or —CR₂₀R₂₀′—;

Y₃, Y₄ and Y₅ are each independently selected from H, hydroxyl, halogen atom, substituted or unsubstituted amino and hydrocarbyloxy;

t₁, t₂ and t₃ are each independently an integer selected from 0-5, preferably, both t₁ and t₂ are 1, t₃ is 0;

R₁₃, R₁₃′ and R₁₄ are each independently selected from —CN, —CF₃, F, —SO₂CF₃, —NO₂, —COOEt, —SO₂ph,

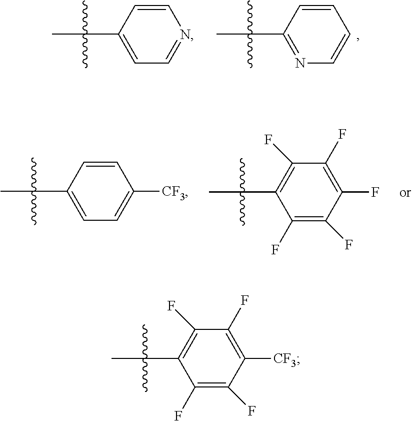 [SMILES: *c1ccncc1 ; *c1ccccn1 ; *c1ccc(C(F)(F)F)cc1 ; *c1c(F)c(F)c(F)c(F)c1F ; *c1c(F)c(F)c(C(F)(F)F)c(F)c1F]

preferably, both R₁₃ and R₁₃′ are —CN, R₁₄ is —CN or

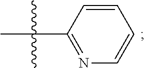 [SMILES: *c1ccccn1]

R₁₅ is —(CH₂)ₘ—,

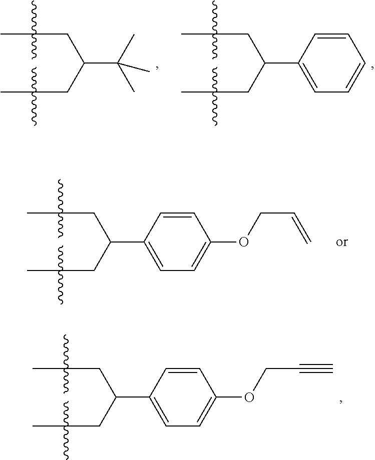

m is an integer of 0-5, preferably m is 3;

R₁₆ and R₁₇ together form one of the following connections:

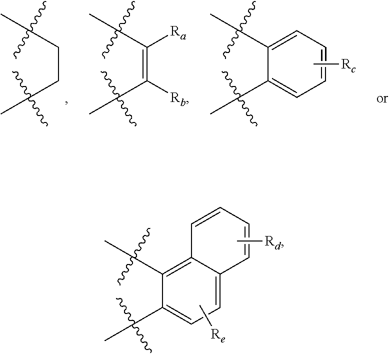

or R₁₆, R₁₇ and X₂ together form a connection

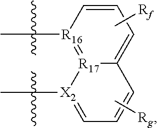

wherein Rₐ, R_{b}, R_{c}, R_{d}, Rₑ, R_{f} and R_{g} are each independently selected from H, halogen, substituted or non-substituted hydrocarbyl, substituted or non-substituted carboxyl, substituted or non-substituted hydroxyl and substituted or non-substituted amino;

R₁₈, R₁₈′, R₁₉, R₂₀ and R₂₀′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxyl, substituted or unsubstituted amido, substituted or unsubstituted ester group and substituted or unsubstituted amino; preferably, R₁₈, R₁₈′ and R₁₉ are each independently selected from H, —(CH₂)_{q}CH₃, —(CH₂)_{q}CF₃, —(CH₂)_{q}CHCH₂, —(CH₂)_{q}CCH, —(CH₂)_{q}OH, —(CH₂)_{q}COOH, —(CH₂)_{q}NH₂, —(CH₂)_{q}CHO, —(CH₂)_{q}CO(CH₂)_{q′}CH₃, —(CH₂)_{q}O(CH₂)_{q′}CH₃,

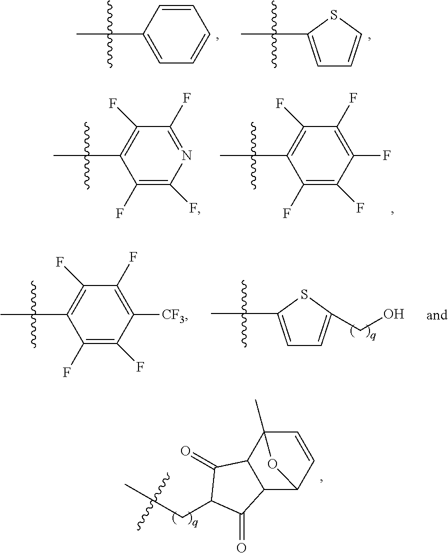

wherein q and q′ are each independently an integer selected from 0-12; preferably, R₁₉ is —CH₂CH₃;

when the group is substituted, the substituent is single or multiple.

In a preferred embodiment,

both Y₃ and Y₅ are H;

Y₄ is Cl, Br or —NR₂₁R₂₁′—;

both t₁ and t₂ are 1, t₃ is 0;

R₂₁ and R₂₁′ are each independently selected from H, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

More preferably, the compound of formula (IV) is Compound II-1, II-2, II-3, II-4, II-5, II-6, II-7, II-8, II-9, II-10, II-11, II-12, II-13, II-14, II-15, II-16 or II-17.

Preferably, the phototherapeutic drug is a photothermal therapeutic drug, a photodynamic therapeutic drug or a photoacoustic therapeutic drug.

The present invention also provides a use of the compound represented by formula (IV), the isomer, pharmaceutically acceptable salt, hydrate or solvate thereof as a photosensitizer. Preferably, the photosensitizer is used to prepare a photothermal therapeutic drug, a photodynamic therapeutic drug or a photoacoustic therapeutic drug.

The present invention also provides a use of the compound represented by formula (IV), the isomer, pharmaceutically acceptable salt, hydrate or solvate thereof in the preparation of a drug for diagnosis and/or treatment of cancer. Preferably, the cancer is esophageal cancer, non-small cell lung cancer, biliary cancer, head and neck cancer, Barrett esophagus, bladder cancer, colorectal cancer, pancreatic cancer, ovarian cancer, prostate cancer, brain tumor, breast cancer or skin cancer; the skin cancer includes melanoma.

The present invention also provides a use of the compound represented by formula (IV), the isomer, pharmaceutically acceptable salt, hydrate or solvate thereof in the preparation of a medicament for the treatment of skin diseases. Preferably, the skin diseases are actinic keratosis, basal cell carcinoma, and skin T-cell lymphoma, Bowen's disease, squamous cell carcinoma, intraepithelial neoplasia of the vulva and anus, or Paget's disease.

The beneficial effects of the present invention:

(1) The invention provides a micro-nano structure formed by the self-assembly of the compound represented by formula (I) or formula (II) in an aqueous solution, a preparation method and application thereof. Experiments have proved that the micro-nano structure has the advantages of high light-to-heat conversion efficiency, good photothermal stability, good photothermal effect and photodynamic effect, easy degradation, and high safety, and can passively target the tumor site. There are broad prospects in diagnosis and treatment of cancer and skin diseases.

(2) The present invention provides a compound represented by formula (III), which can self-assemble into a micro-nano structure in an aqueous solution, and thus has the advantage of high light-to-heat conversion efficiency, good photothermal stability, good photothermal effect and photodynamic effect, easy degradation, and high safety.

(3) The invention also provides a use of the compound represented by formula (IV) for the preparation drugs for phototherapy, or drugs for diagnosis and treatment of cancer, or drugs for the treatment of skin diseases, which has good therapeutic effect, less trauma, and has great market value and broad economy prospect.

### DESCRIPTION OF FIGURES

FIG. 1 shows the schematic structural diagram of the disclosed organic small molecule fluorescent compound ICG, IR808, IR825 and IR780;

FIG. 2 shows the synthesis route of organic small molecule fluorescent Compound II-1 of the present invention;

FIG. 3 shows the ultraviolet absorption spectrum and fluorescence emission spectrum of Compound II-1 in different polar solvents;

FIG. 4 shows the ultraviolet absorption spectrum of Compounds II-16, II-17 and II-18 in water and in organic solvents;

FIG. 5 shows the transmission electron microscope images, cryo-transmission electron microscope images, atomic force microscope images and data of nano-sheet formed by self-assembly of Compound II-1 in aqueous solution; crystal structure of II-1 and simulated schematic structural diagram of the nano-sheet;

FIG. 6 shows the particle size test result of an uncharged organic small molecule fluorescent compound of the present invention in an aqueous solution, showing that the uncharged organic small molecule fluorescent compound can self-assemble into a micro-nano structure;

FIG. 7 shows the dynamic light scattering DLS of Compound II-16 to II-18;

FIG. 8 shows the temperature change diagram and photothermal stability of Compound II-1 under 808 nm laser irradiation;

FIG. 9 shows the temperature change diagram of Compounds II-16, II-17 and II-18 at different concentrations under 808 nm laser irradiation;

FIG. 10 shows the photothermal conversion efficiency of Compounds II-16, II-17 and II-18;

FIG. 11 shows the photothermal stability of Compound II-16;

FIG. 12 shows the photothermal stability of Compound II-17;

FIG. 13 shows the assembly stability of Compound II-16 in different medium;

FIG. 14 shows the microscopic image of the assembled micro-nano structure of Compound II-1 phagocytosed by cells, showing that the assembled micro-nano structure is located in the lysosome of the cell;

FIG. 15 shows the infrared fluorescence intensity changes of the tumor site at different time points after the micro-nano structure assembled by Compound II-1 was injected intravenously into mice with subcutaneous metastases;

FIG. 16 shows the infrared fluorescence imaging of the whole body of mice at different time points after the micro-nano structure assembled by Compound II-1 was injected intravenously into mice with subcutaneous metastases;

FIG. 17 shows the photothermal imaging of the micro-nano structure assembled by Compound II-1 in photothermal therapy in mice with subcutaneous metastases;

FIG. 18 shows the changes in tumor volume of mice with subcutaneous metastases after intravenous injection of the micro-nano structure assembled by Compound II-1 and photothermal treatment;

FIG. 19 shows the changes in body weight of mice with subcutaneous metastases after intravenous injection of the micro-nano structure assembled by Compound II-1 and photothermal treatment;

FIG. 20 shows the results of photoacoustic imaging test of Compound II-1 in mice with subcutaneous metastases;

FIG. 21 shows the establishment of mouse sentinel lymphatic metastasis model;

FIG. 22 shows the infrared fluorescence intensity changes of the tumor site at different time points after the micro-nano structure assembled by Compound II-1 was injected into the sentinel lymphatic metastasis mice via tail vein injection and intratumoral injection;

FIG. 23 shows the infrared fluorescence imaging of the control group at different time points with saline plus laser irradiation after the micro-nano structure assembled by Compound II-1 enters sentinel lymphatic metastasis mice in different ways;

FIG. 24 shows the infrared fluorescence imaging of the experimental group of intratumoral injection and laser irradiation at different time points after the micro-nano structure assembled by Compound II-1 enters sentinel lymphatic metastasis mice in different ways;

FIG. 25 shows the infrared fluorescence imaging of the experimental group at different time points after tail vein injection and laser irradiation after the micro-nano structure assembled by Compound II-1 enters sentinel lymphatic metastasis mice in different ways;

FIG. 26 shows the comparison of the temperature changes of sentinel lymph nodes after laser irradiation after the micro-nano structure assembled by Compound II-1 enters sentinel lymphatic metastasis mice in different ways;

FIG. 27 shows the pictures after photothermal treatment of sentinel lymphatic metastasis mice by Compound II-1 in different ways;

FIG. 28 shows the changes in body weight of mice with sentinel lymphoma metastasis during photothermal treatment by Compound II-1 in different ways;

FIG. 29 shows the pictures of the mice stained with ink in lung on day 20 after photothermal treatment of sentinel lymphatic metastasis mice by Compound II-1 in different ways;

FIG. 30 shows fluorescence imaging in mice with subcutaneous metastases of Compound II-16 to II-18;

FIG. 31 shows imaging of internal organs of mice with subcutaneous metastases of compound II-16 to II-18;

FIG. 32 shows experimental results of Compound II-1 photodynamic activity test;

FIG. 33 shows experimental results of photoacoustic imaging test of Compound II-1 in aqueous solution.

### DETAILED DESCRIPTION OF THE INVENTION

The methods and techniques of the present invention are generally carried out in accordance with conventional methods known in the art, unless otherwise stated. Nomenclature and experimental methods and techniques related to biology, pharmacology, and medical and medicinal chemistry described herein are known and commonly used in the art. Standard techniques are used for chemical synthesis, chemical analysis, pharmaceutical manufacturing, formulation and delivery, and detection or testing methods.

The scientific and technical terms used herein should be understood from the meanings of those skilled in the art, unless otherwise stated. However, the following terms have the following definitions:

The term “micro-nano structure” refers to a tiny structure with a size of less than 500 nm, that is, a new system that is constructed or assembled according to a certain rule, with a compound having the structure of formula (I) or formula (II) as the basic unit. It includes one-dimensional, two-dimensional, and three-dimensional systems. The specific forms of micro-nano structures can include particles, wires, rods, columns, tubes, whiskers, fibers, ribbons, filaments, vesicles, etc., or a combination form of the above forms. Preferably, the micro-nano structure is a nano-sheet; more preferably, the micro-nano structure is a columnar nano-sheet; more preferably, the micro-nano structure is a cylindrical nano-sheet.

The term “isomer” includes conformational isomers, optical isomers (such as enantiomers and diastereomers) and geometric isomers (such as cis-trans isomers). These isomers or combinations thereof can be used as racemic mixtures (racemates), individual enantiomers, individual diastereomers, mixtures of diastereomers, cis or trans isomers.

The term “aqueous solution” refers to a liquid mixture containing water, and the weight percentage of water in the aqueous solution is 0.1%-100%, preferably 1%-100%, more preferably 10-100%. The aqueous solution may be a uniform and stable mixture formed by the mutual dissolution of the components, or an uneven, unstable mixture formed by the insolubilization of the components, such as a suspension or an emulsion. Specific examples of the aqueous solution in the present invention may include: physiological saline, plasma, phosphate buffered saline (PBS), glycine-hydrochloric acid buffer, citric acid-sodium hydroxide-hydrochloric acid buffer, citric acid-sodium citrate buffer, acetic acid-sodium acetate buffer, barbiturate buffer, trishydroxymethylaminomethane (Tris) buffer and boric acid-borax buffer, etc.

The term “conjugated carbon chain” refers to a molecular structure containing two or more carbon-carbon double bonds, and the double bonds and single bonds are alternately arranged with each other.

The term “pharmaceutically acceptable salts” refers to salts formed by the reaction of the above compounds with inorganic acids, organic acids, alkali metals or alkaline earth metals. These salts include (but are not limited to): (1) salts with inorganic acids such as hydrochloric acid, hydrobromic acid, hydroiodic acid, sulfuric acid, nitric acid, phosphoric acid; (2) salts with organic acids such as acetic acid, lactic acid, citric acid, succinic acid, fumaric acid, gluconic acid, benzoic acid, methanesulfonic acid, ethanesulfonic acid, benzenesulfonic acid, p-toluenesulfonic acid, oxalic acid, succinic acid, tartaric acid, maleic acid, or arginine; (3) other salts, including salts formed with alkali metals or alkaline earth metals (such as sodium, potassium, calcium or magnesium), ammonium salts or water-soluble amine salts (such as N-methylglucamine salt), lower alkanol ammonium salts and other pharmaceutically acceptable amine salts (such as methylamine salt, ethylamine salt, propylamine salt, dimethylamine salt, trimethylamine salt, diethylamine salt, triethylamine salt, tert-butylamine salt, ethylenediamine salt, hydroxyethylamine salt, dihydroxyethylamine salt, trihydroxyethylamine salt, and amine salts formed from morpholine, piperazine, and lysine, respectively, or other conventional “precursor drug” forms.

The precursor refers to a compound which is metabolized or chemically reacted in the patient's body after being taken by an appropriate method to transform into a compound included in the general formula of the present invention, and a salt or solution composed of the compound. Precursors of compounds include, but are not limited to, carboxylates, carbonates, phosphates, nitrates, sulfates, sulfones, sulfoxides, amino compounds, carbamates, azo compounds, phosphoramides, glucoside, ether, acetal and other forms of the compounds.

The term “halogen atom” refers to any radio-stable atom in column 7 of the periodic table, i.e. fluorine, chlorine, bromine or iodine, preferably fluorine and chlorine.

The term “hydrocarbyl” refers to any linear or branched, substituted or unsubstituted saturated hydrocarbon group, wherein the hydrocarbon group having 1 to 10 carbon atoms includes, but is not limited to, methyl, ethyl, n-propyl, isopropyl, cyclopropyl, n-butyl, isobutyl, sec-butyl, tert-butyl, n-pentyl, isopentyl, neopentyl, tert-pentyl, 2,4,4-trimethylpentyl, cyclopentyl, n-hexyl, isohexyl, cyclohexyl, n-heptyl, cycloheptyl, n-octyl, 2-ethylhexyl, cyclooctyl, n-nonyl, cyclononyl or n-decyl. Other long-chain alkyl groups with more carbon atoms include but are not limited to squalene, nonadecanol and the like.

The terms “aryl”, “substituted aryl”, “heteroaryl” and “substituted heteroaryl” refer to aromatic hydrocarbon rings, preferably having 5, 6 or 7 atoms, most preferably having 6 atoms to form the ring. “Heteroaryl” and “substituted heteroaryl” refer to aromatic hydrocarbon rings having at least one heteroatom (e.g., oxygen, sulfur, or nitrogen atom) and at least one carbon atom in the ring.

The term “substituted” refers to any group in which at least one hydrogen atom is replaced by a substituent selected from halogen atom, alkyl, substituted alkyl, alkoxy, substituted alkoxy, cyclic hydrocarbyl, substituted cyclic hydrocarbyl, cycloalkenyl, substituted cycloalkenyl, acyl, acylamino, acyloxy, amino, substituted amino, aminoacyl, aminoacyloxy, oxyacylamino, cyano, hydroxyl, carboxyl, carboxyalkyl, keto, thioketo, thiol, thioalkoxy, substituted thioalkoxy, aryl, aryloxy, heteroaryl, heteroaryloxy, heterocyclyl, heterocyclyloxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “particle size” refers to the size of particles, also known as “granularity” or “diameter”. When a certain physical characteristic or physical behavior of the measured particle is closest to a homogeneous sphere (or combination) of a certain diameter, the diameter (or combination) of the sphere is taken as the equivalent particle size of the measured particle. The particle size parameter of the micro-nano structure of the present invention is measured by the principle of dynamic light scattering (DLS), specifically measured by a laser particle size analyzer.

The term “therapeutically effective dose” refers to any amount of the drug as described below. When used alone or in combination with another therapeutic agent, the amount of the drug can promote the regression of the disease, which manifests as a reduction in the severity of the symptoms of the disease, an increase of the frequency and duration of the disease-free symptom period, or the prevention of the disorder or disability caused by the disease. The “therapeutically effective dose” of the drug of the present invention also includes the “prophylactically effective dose”. The “prophylactically effective dose” is any amount of the drug as described below, when the amount of the drug is administered alone or in combination with another therapeutic agent to a subject having a risk of developing a disease or suffering from a disease recurrence, the occurrence or recurrence of the disease can be suppressed.

As will be apparent to those skilled in the art, the effective in vivo dosage and specific mode of administration will vary according to the type, weight and age of the mammal being treated, the specific compounds used and the specific purpose of using these compounds. Those skilled in the art can determine the effective dose level (i.e., the dose level necessary to achieve the desired effect) according to conventional pharmacological methods. Generally, the human clinical application of the product starts at a lower dose level and then continuously increases the dose level until the desired effect is achieved. Alternatively, acceptable in vitro studies can be used by existing pharmacological methods to establish useful doses and routes of administration of the compositions identified by this method.

The term “cancer” refers to a large class of diseases characterized by the uncontrolled growth of abnormal cells in the body. Uncontrolled cell division and growth division and growth result in the formation of malignant tumors or cells that invade adjacent tissues and can also be transferred to the distal part of the body through the lymphatic system or blood flow. In the present invention, another equivalent description of “treatment of cancer” is “treatment of tumor” or “anti-cancer” or “anti-tumor”.

The “diagnostic agent” is any chemical substance used for diagnosis. For example, diagnostic agents include imaging agents such as those containing radioactive isotopes such as indium or technetium; contrast agents containing iodine or gadolinium; enzymes such as horseradish peroxidase, GFP, alkaline phosphatase, or β-Galactosidase; fluorescent substances, such as europium derivatives; luminescent substances, such as N-methyl acridine derivatives, etc.

The “therapeutic agent” is any chemical substance recognized in the art as a biological, physiological or pharmacologically active substance. Therapeutic agents are also referred to as “drugs”, examples of which are described in known references (such as Merck Index, Physicians Desk Reference, and The Pharmacological Basics of therapeutics), and they include (but are not limited to) drugs, vitamins, minerals substance supplements, substances used to treat, prevent, diagnose, cure or alleviate a disease or illness, substances or prodrugs that affect the structure or function of the body having biological activity or being more active when being placed in a physiological environment. Various forms of therapeutic drugs can be used, wherein when administered to a subject, the composition can be released from the subject into adjacent tissues or fluids.

The “targeting molecule” is any molecule capable of guiding the micro-nano structure to a specific target, for example, by binding a receptor or other molecule on the surface of the target cell. The targeting molecule can be a protein, peptide, nucleic acid molecule, sugar or polysaccharide, receptor ligand, or other small molecule. The degree of specificity can be adjusted by selecting targeting molecules. For example, antibodies usually show higher specificity. Antibodies can be polyclonal, monoclonal, fragment, recombinant, or single chain, many of which are commercially available or can be easily obtained using standard techniques.

Some examples of the compounds of the invention are shown in Table 1, Compounds II-1 to II-46:

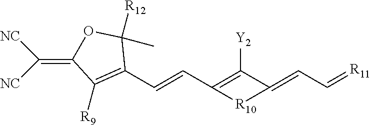

TABLE 1
Examples of compounds of the Invention

| Compound | | | | | |
|---|---|---|---|---|---|
| number | R₉ | Y₂ | R₁₀ | R₁₁ | R₁₂ |
| II-1 | —CN | —Cl | —(CH₂)₃— | 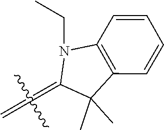 [SMILES: *=C1N(CC)c2ccccc2C1(C)C] | —CH₃ |
| II-2 | —CN | —Cl | —(CH₂)₃— | 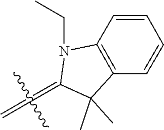 [SMILES: *=C1N(CC)c2ccccc2C1(C)C] | 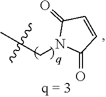 |
| II-3 | —CN | —Cl | —(CH₂)₃— | 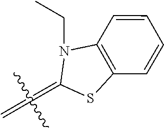 [SMILES: *=C1Sc2ccccc2N1CC] | 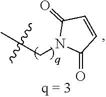 |
| II-4 | —CN | —Cl | —(CH₂)₃— | 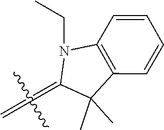 [SMILES: *=C1N(CC)c2ccccc2C1(C)C] | 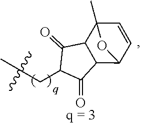 |
| II-5 | —CN | —Cl | —(CH₂)₃— | 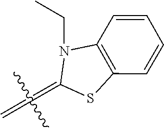 [SMILES: *=C1Sc2ccccc2N1CC] | 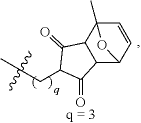 |
| II-6 | —CN | —Cl | —(CH₂)₃— | 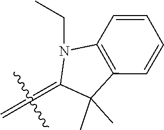 [SMILES: *=C1N(CC)c2ccccc2C1(C)C] | —(CH₂)₃OH |
| II-7 | —CN | —Cl | —(CH₂)₃— | 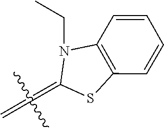 [SMILES: *=C1Sc2ccccc2N1CC] | —(CH₂)₃OH |
| II-8 | —CN | —Cl | —(CH₂)₃— | 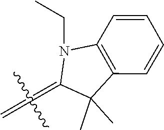 [SMILES: *=C1N(CC)c2ccccc2C1(C)C] | —CF₃ |
| II-9 | —CN | —Cl | —(CH₂)₃— | 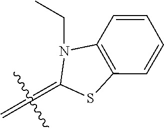 [SMILES: *=C1Sc2ccccc2N1CC] | —CF₃ |
| II-10 | 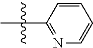 [SMILES: *c1ccccn1] | —Cl | —(CH₂)₃— | 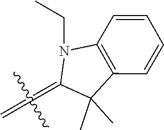 [SMILES: *=C1N(CC)c2ccccc2C1(C)C] | —CH₃ |
| II-11 | 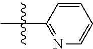 [SMILES: *c1ccccn1] | —Cl | —(CH₂)₃— | 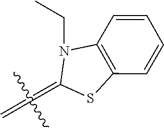 [SMILES: *=C1Sc2ccccc2N1CC] | —CH₃ |
| II-12 | —CN | —Cl | 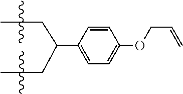 [SMILES: *CC(C*)c1ccc(OCC=C)cc1] | 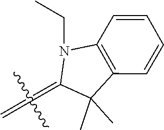 [SMILES: *=C1N(CC)c2ccccc2C1(C)C] | —CH₃ |
| II-13 | —CN | —Cl | 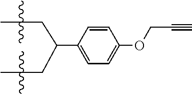 [SMILES: *CC(C*)c1ccc(OCC#C)cc1] | 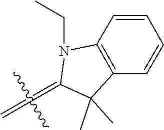 [SMILES: *=C1N(CC)c2ccccc2C1(C)C] | —CH₃ |
| II-14 | —CN | —Cl | —(CH₂)₃— | 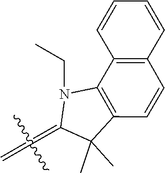 [SMILES: *=C1N(CC)c2c(ccc3ccccc23)C1(C)C] | —CH₃ |
| II-15 | —CN | 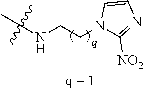 | 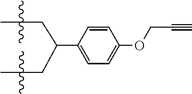 [SMILES: *CC(C*)c1ccc(OCC#C)cc1] | 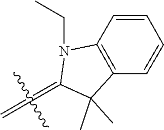 [SMILES: *=C1N(CC)c2ccccc2C1(C)C] | —CH₃ |
| II-16 | —CN | —Cl | —(CH₂)₃— | 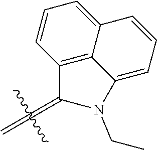 [SMILES: *=c1c2cccc3cccc(c32)n1CC] | —CH₃ |
| II-17 | —CN | —Cl | —(CH₂)₃— | 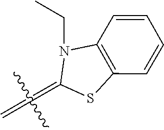 [SMILES: *=C1Sc2ccccc2N1CC] | —CH₃ |
| II-18 | —CN | —Cl | —(CH₂)₃— | 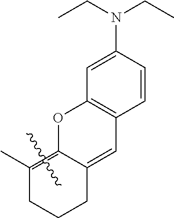 | —CH₃ |
| II-19 | —CN | —Cl | —(CH₂)₃— | 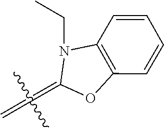 [SMILES: *=C1Oc2ccccc2N1CC] | —CH₃ |
| II-20 | —CN | —Cl | —CH₂— | 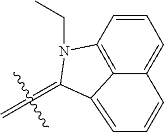 [SMILES: *=c1c2cccc3cccc(c32)n1CC] | —CH₃ |
| II-21 | —CN | —Cl | —CH₂— | 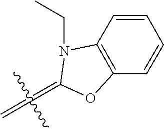 [SMILES: *=C1Oc2ccccc2N1CC] | —CH₃ |
| II-22 | —CN | —Cl | —CH₂— | 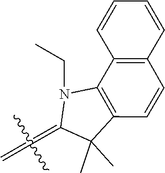 [SMILES: *=C1N(CC)c2c(ccc3ccccc23)C1(C)C] | —CH₃ |
| II-23 | —CN | —Cl | —CH₂— | 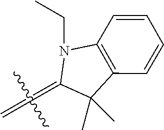 [SMILES: *=C1N(CC)c2ccccc2C1(C)C] | —CH₃ |
| II-24 | —CN | —Cl | —CH₂— | 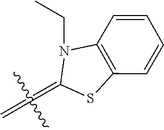 [SMILES: *=C1Sc2ccccc2N1CC] | —CH₃ |
| II-25 | —CN | —Cl | —(CH₂)₅— | 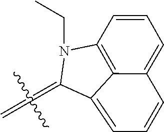 [SMILES: *=c1c2cccc3cccc(c32)n1CC] | —CH₃ |
| II-26 | —CN | —Cl | —(CH₂)₅— | 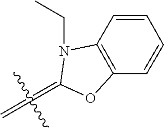 [SMILES: *=C1Oc2ccccc2N1CC] | —CH₃ |
| II-27 | —CN | —Cl | —(CH₂)₅— | 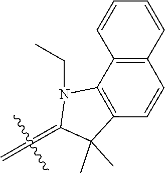 [SMILES: *=C1N(CC)c2c(ccc3ccccc23)C1(C)C] | —CH₃ |
| II-28 | —CN | —Cl | —(CH₂)₅— | 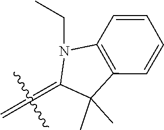 [SMILES: *=C1N(CC)c2ccccc2C1(C)C] | —CH₃ |
| II-29 | —CN | —Cl | —(CH₂)₅— | 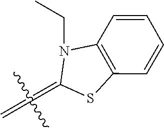 [SMILES: *=C1Sc2ccccc2N1CC] | —CH₃ |
| II-30 | —CN | —NHCH₃ | —CH2— | 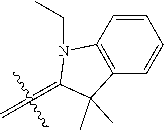 [SMILES: *=C1N(CC)c2ccccc2C1(C)C] | —CH₃ |
| II-31 | —CN | —NHCH₃ | —(CH₂)₃— | 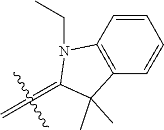 [SMILES: *=C1N(CC)c2ccccc2C1(C)C] | —CH₃ |
| II-32 | —CN | —NHCH₃ | —(CH₂)₅— | 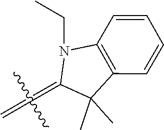 [SMILES: *=C1N(CC)c2ccccc2C1(C)C] | —CH₃ |
| II-33 | —CN | —Cl | —CH₂— | 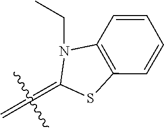 [SMILES: *=C1Sc2ccccc2N1CC] | —(CH₂)₃OH |
| II-34 | —CN | —Cl | —(CH₂)₅— | 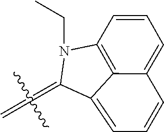 [SMILES: *=c1c2cccc3cccc(c32)n1CC] | —(CH₂)₃OH |
| II-35 | —CN | —Cl | —(CH₂)₅— | 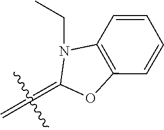 [SMILES: *=C1Oc2ccccc2N1CC] | —(CH₂)₃OH |
| II-36 | —CN | —Cl | —(CH₂)₅— | 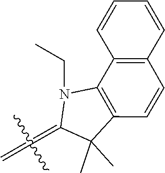 [SMILES: *=C1N(CC)c2c(ccc3ccccc23)C1(C)C] | —(CH₂)₃OH |
| II-37 | —CN | —Cl | —(CH₂)₅— | 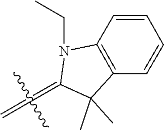 [SMILES: *=C1N(CC)c2ccccc2C1(C)C] | —(CH₂)₃OH |
| II-38 | —CN | —Cl | —(CH₂)₅— | 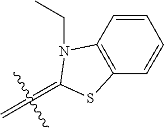 [SMILES: *=C1Sc2ccccc2N1CC] | —(CH₂)₃OH |
| II-39 | —CN | —NHCH₃ | —CH2— | 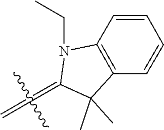 [SMILES: *=C1N(CC)c2ccccc2C1(C)C] | —(CH₂)₃OH |
| II-40 | —CN | —NHCH₃ | —(CH₂)₃— | 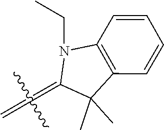 [SMILES: *=C1N(CC)c2ccccc2C1(C)C] | —(CH₂)₃OH |
| II-41 | —CN | —NHCH₃ | —(CH₂)₅— | 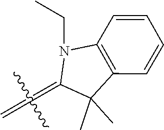 [SMILES: *=C1N(CC)c2ccccc2C1(C)C] | —(CH₂)₃OH |
| II-42 | —CN | —NHCH₃ | —(CH₂)₃— | 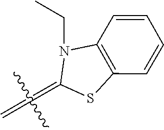 [SMILES: *=C1Sc2ccccc2N1CC] | —(CH₂)₃OH |
| II-43 | —CN | —NHCH₃ | —(CH₂)₃— | 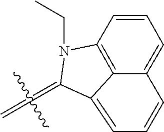 [SMILES: *=c1c2cccc3cccc(c32)n1CC] | —(CH₂)₃OH |
| II-44 | —CN | —NHCH₃ | —(CH₂)₃— | 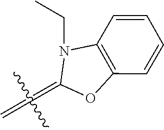 [SMILES: *=C1Oc2ccccc2N1CC] | —(CH₂)₃OH |
| II-45 | —CN | —NHCH₃ | —(CH₂)₃— | 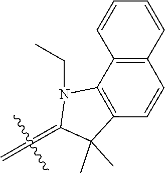 [SMILES: *=C1N(CC)c2c(ccc3ccccc23)C1(C)C] | —(CH₂)₃OH |
| II-46 | —CN | —OCH₃ | —(CH₂)₃— | 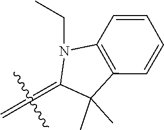 [SMILES: *=C1N(CC)c2ccccc2C1(C)C] | —CH₃ |

The above compounds (except II-18) can be synthesized by the following reaction formula, and the reaction formula for the synthesis of II-18 will be further explained in Example 2.

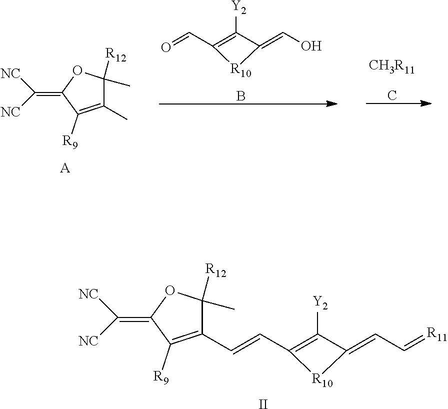

The main synthetic steps include:
1. Provide Compounds A, B and C respectively;

### Synthesis of Compound A

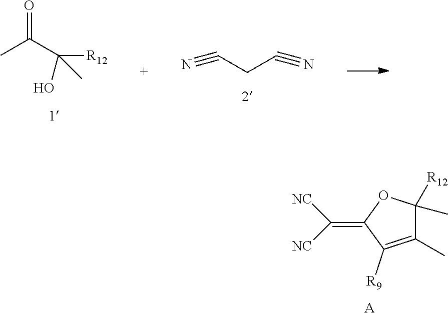

Compound 1′ and Compound 2′ and ethanol magnesium were dissolved in ethanol, and reacted at 60° C. for 24 hours. The solvent was removed by evaporation under vacuum, and the resulting solid was purified by column chromatography to obtain the target Compound A.

### Synthesis of Compound B

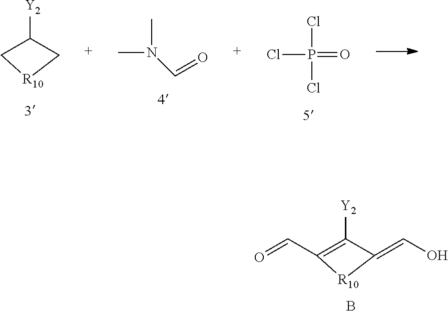

Dichloromethane and Compound 4′ were added to the flask under ice bath and stirred. Compound 5′ was added under constant pressure and stirred. Compound 3′ was added and reacted at 80° C. for 3 hours. After the reaction was completed, the product was poured into the brittle ice to quench the reaction, placed overnight in the refrigerator. The solvent was removed by evaporation under vacuum to give crude product Compound B, which was directly used in the next reaction without purification.

### Synthesis of Compound C

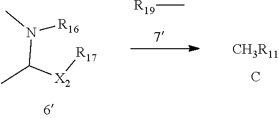

Compound 6′ and Compound 7′ were added to acetonitrile. The mixture was heated to 110° C. and refluxed for 24 hours. The solvent was removed by evaporation under vacuum, and the resulting solid was washed 3 times with diethyl ether to obtain a Compound C.
2. Compound A and Compound B were dissolved in ethanol, heated to reflux, and then Compound C was added, heated to reflux, the solvent was removed by evaporation under vacuum, and the resulting solid was purified by column chromatography to obtain the target Compound II.

The present invention will be further described below with reference to the examples.

### Example 1 Synthesis of Compound II-1 and its Fluorescence Properties

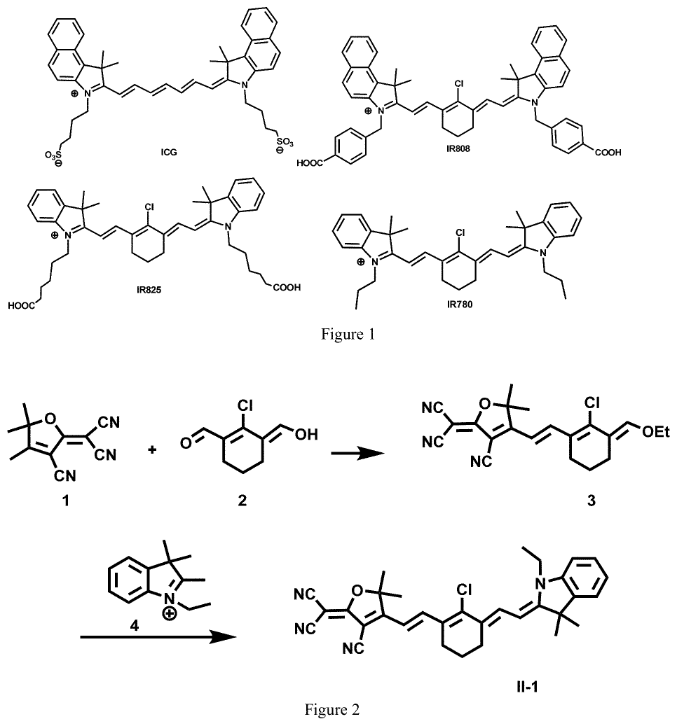

As shown in FIG. 2, the synthesis of Compound II-1 includes the following steps:

1) Synthesis of Compound 1: 0.97 g of malononitrile and 0.62 g of ethanol magnesium were added to 10 mL of ethanol, and 0.5 mL of 3-hydroxy-3-methylbutane-2-one was added. Heated to 60° C. and reacted for 12 hours. The solvent was removed by evaporation under vacuum, and the resulting solid was purified by column chromatography to obtain the target Compound 1. ¹H NMR (400 MHz, CDCl₃): δ (ppm): 2.36 (s, 3H), 1.63 (s, 6H). 2) Synthesis of Compound 2: 20 mL of dichloromethane and 20 mL DMF were added to the flask under ice bath and stirred, and 17.5 mL of phosphorus oxychloride was added at constant pressure and stirred, and then 5.3 mL of cyclohexanone was added, heated to 80° C. and reacted for 3 hours. After the reaction was completed, the product was poured into the brittle ice to quench the reaction, placed overnight in the refrigerator. The solvent was removed by evaporation under vacuum to obtain the crude Compound 2, which was directly used in the next reaction without purification.

3) Synthesis of Compound 4: 5 g of 2,3,3-trimethyl-3H-indole and 6 g of iodoethane were added to 20 mL of acetonitrile. The mixture was heated to 110° C. and refluxed for 24 hours. The solvent was removed by evaporation under vacuum, and the resulting solid was washed 3 times with diethyl ether to obtain a Compound 4.

4) Synthesis of Compound II-1: 3.0 g of Compound 1 and 1.99 g of Compound 2 were dissolved in 50 mL of ethanol, heated to 100° C. and refluxed for 4 hours, then 2.75 g of Compound 4 was added, heated and refluxed for 6 hours. The solvent was removed by evaporation under vacuum, and the resulting solid was purified by column chromatography to obtain the target Compound II-1. ¹H NMR (400 MHz, CDCl₃): δ (ppm): 8.13 (d, 1H), 7.18 (d, 1H), 7.30 (t, 2H), 7.07 (t, 1H), 6.85 (d, 1H), 6.34 (d, 1H), 5.72 (d, 1H), 3.87 (d, 2H), 2.61 (d, 4H), 1.90 (m, 2H), 1.75 (s, 6H), 1.66 (s, 6H), 1.35 (t, 3H).

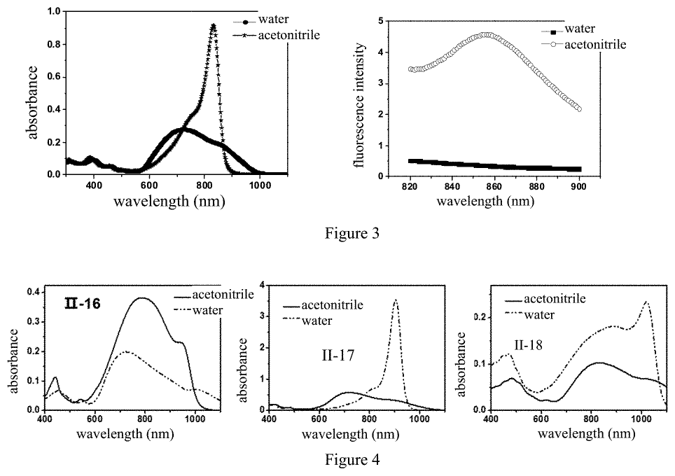

The ultraviolet absorption spectrum and fluorescence emission spectrum of Compound II-1 in water and acetonitrile are shown in the left and right diagrams of FIG. 3, respectively. It can be seen that the absorption and emission spectra of Compound II-1 in the two solvents are significantly different, among which the absorption spectrum of II-1 in aqueous solution is wider and the emitted light is less. It is suggested that the compounds of the present invention have different physical properties in aqueous solutions and organic solvents.

### Example 2 Synthesis of Compound II-2 to II-18

Compounds II-2 to II-18 can be prepared by the method similar to that of Example 1.

### 1. Synthesis of Compound II-2

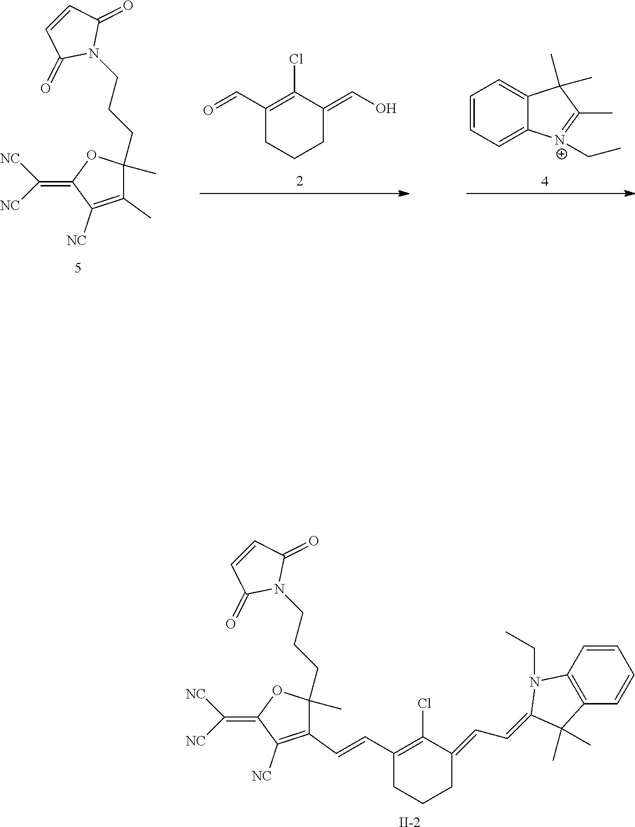

Compound II-2 was prepared by using Compound 5 to replace Compound 1 in Example 1, and the remaining required reagents and preparation methods used were the same as step 4) in Example 1.

1H NMR (400 MHz, CDCl3): δ (ppm): 8.60-8.59 (d, 2H), 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 3.87-3.72 (q, 2H), 3.20-3.18 (t, 2H), 2.60-2.57 (t, 4H), 1.91-1.88 (m, 2H), 1.75 (s, 3H), 1.66 (s, 6H), 1.56-1.53 (m, 4H), 1.36-1.33 (t, 3H).

### 2. Synthesis of Compound II-3

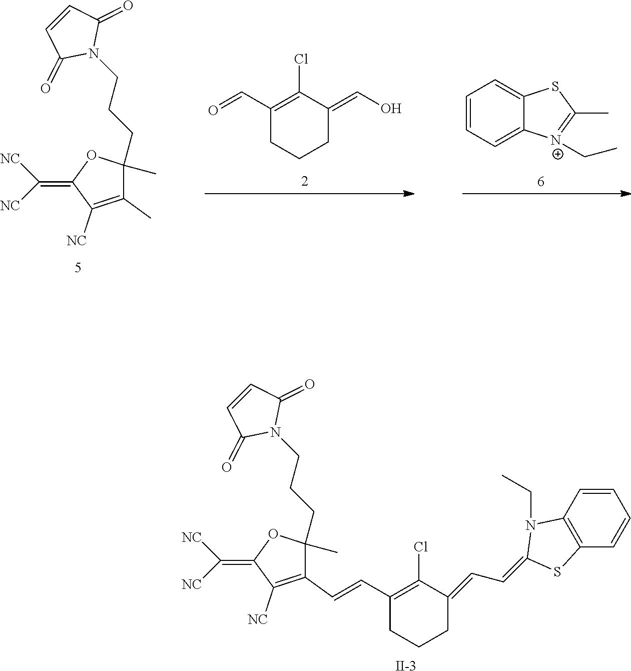

Compound II-3 was prepared by using Compound 5 and Compound 6 to replace Compound 1 and Compound 4 in Example 1, respectively, and the remaining required reagents and preparation methods used were the same as step 4) in Example 1.

¹H NMR (400 MHz, CDCl₃): δ (ppm): 8.60-8.59 (d, 2H), 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 3.87-3.72 (q, 2H), 3.20-3.18 (t, 2H), 2.60-2.57 (t, 4H), 1.91-1.88 (m, 2H), 1.75 (s, 3H), 1.56-1.53 (m, 4H), 1.36-1.33 (t, 3H).

### 3. Synthesis of Compound II-4

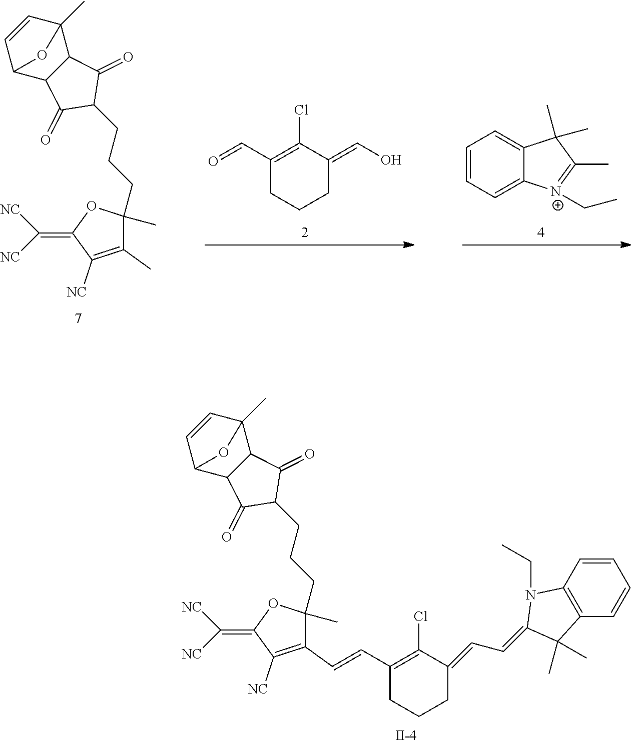

Compound II-4 was prepared by using Compound 7 to replace Compound 1 in Example 1, and the remaining required reagents and preparation methods used were the same as step 4) in Example 1.

¹H NMR (400 MHz, CDCl₃): δ (ppm): 8.60-8.59 (d, 2H), 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 4.55 (s, 1H), 3.87-3.72 (q, 2H), 3.20-3.18 (t, 2H), 3.12 (t, 1H), 2.96-2.94 (t, 2H), 2.60-2.57 (t, 4H), 1.91-1.88 (m, 2H), 1.82-1.81 (s, 3H), 1.75 (s, 3H), 1.66 (s, 6H), 1.56-1.53 (m, 4H), 1.36-1.33 (t, 3H).

### 4. Synthesis of Compound II-5

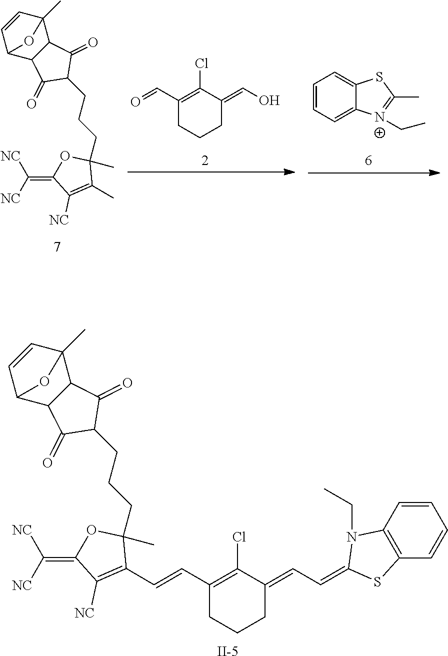

Compound II-5 was prepared by using Compound 7 and Compound 6 to replace Compound 1 and Compound 4 in Example 1, respectively, and the remaining required reagents and preparation methods used were the same as step 4) in Example 1.

¹H NMR (400 MHz, CDCl₃): δ (ppm): 8.60-8.59 (d, 2H), 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 4.55 (s, 1H), 3.87-3.72 (q, 2H), 3.20-3.18 (t, 2H), 3.12 (t, 1H), 2.96-2.94 (t, 2H), 2.60-2.57 (t, 4H), 1.91-1.88 (m, 2H), 1.82-1.81 (s, 3H), 1.75 (s, 3H), 1.56-1.53 (m, 4H), 1.36-1.33 (t, 3H).

### 5. Synthesis of Compound II-6

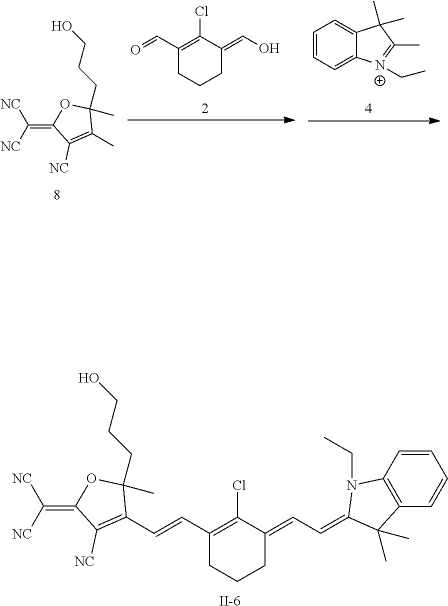

Compound II-6 was prepared by using Compound 8 to replace Compound 1 in Example 1, and the remaining required reagents and preparation methods used were the same as step 4) in Example 1

¹H NMR (400 MHz, CDCl₃): δ (ppm): 8.17-8.16 (t, 1H), 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 3.87-3.72 (q, 2H), 3.20-3.18 (t, 2H), 2.60-2.57 (t, 4H), 1.91-1.88 (m, 2H), 1.75 (s, 3H), 1.66 (s, 6H), 1.56-1.53 (m, 4H), 1.36-1.33 (t, 3H).

### 6. Synthesis of Compound II-7

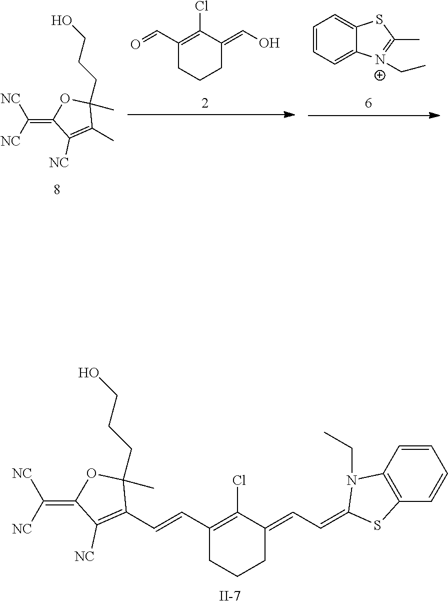

Compound II-7 was prepared by using Compound 8 and Compound 6 to replace Compound 1 and Compound 4 in Example 1, respectively, and the remaining required reagents and preparation methods used were the same as step 4) in Example 1.

¹H NMR (400 MHz, CDCl₃): δ (ppm): 8.17-8.16 (t, 1H), 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 3.87-3.72 (q, 2H), 3.20-3.18 (t, 2H), 2.60-2.57 (t, 4H), 1.91-1.88 (m, 2H), 1.75 (s, 3H), 1.56-1.53 (m, 4H), 1.36-1.33 (t, 3H).

### 7. Synthesis of Compound II-8

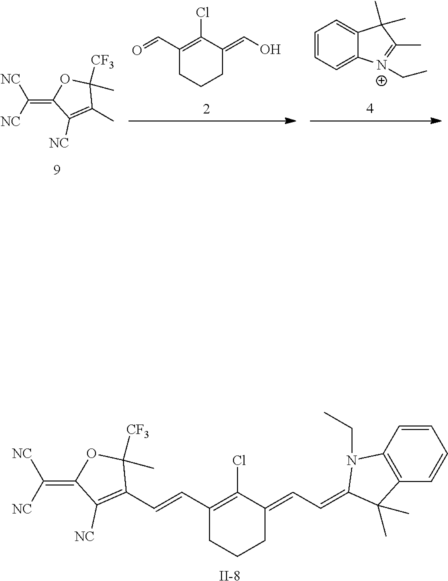

Compound II-8 was prepared by using Compound 9 to replace Compound 1 in Example 1, and the remaining required reagents and preparation methods used were the same as step 4) in Example 1.

¹H NMR (400 MHz, CDCl₃): δ (ppm): 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 3.87-3.72 (q, 2H), 2.60-2.57 (t, 4H), 2.33-2.32 (s, 3H), 1.91-1.88 (m, 2H), 1.66 (s, 6H), 1.36-1.33 (t, 3H).

### 8. Synthesis of Compound II-9

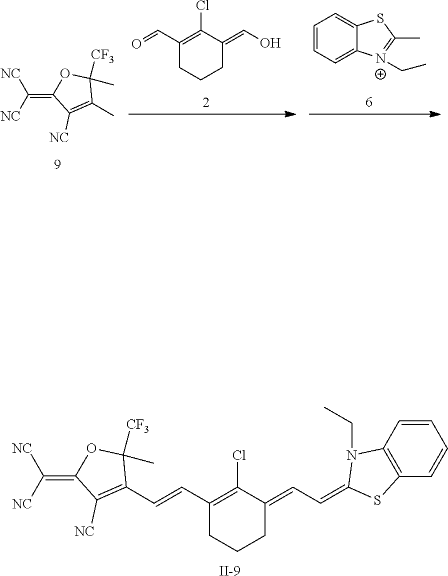

Compound II-9 was prepared by using Compound 9 and Compound 6 to replace Compound 1 and Compound 4 in Example 1, respectively, and the remaining required reagents and preparation methods used were the same as step 4) in Example 1.

¹H NMR (400 MHz, CDCl₃): δ (ppm): 8.13-8.10 (d, 1H), 7.98-7.96 (d, 1H), 7.30-7.28 (d, 2H), 7.08-7.05 (t, 1H), 6.86-6.85 (d, 1H), 6.36-6.33 (d, 1H), 5.71-5.69 (d, 1H), 3.87-3.72 (q, 2H), 2.60-2.57 (t, 4H), 2.33-2.32 (s, 3H), 1.91-1.88 (m, 2H), 1.36-1.33 (t, 3H).

### 9. Synthesis of Compound II-10

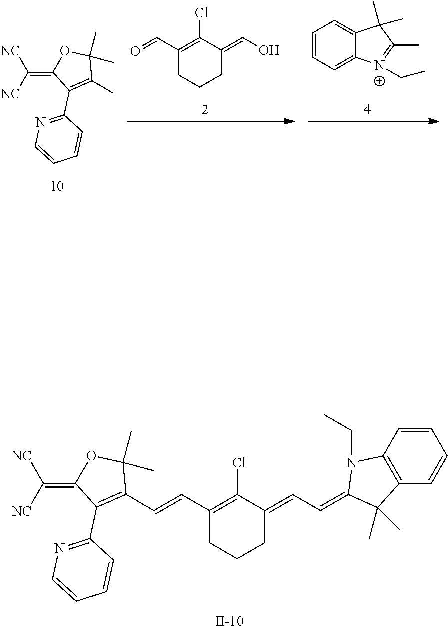

Compound II-10 was prepared by using Compound 10 to replace Compound 1 in Example 1, and the remaining required reagents and preparation methods used were the same as step 4) in Example 1.

¹H NMR (400 MHz, CDCl₃): δ (ppm): 8.75-8.71 (m, 1H), 8.13-8.10 (d, 1H), 7.98-7.96 (d, 1H), 7.85-7.76 (m, 1H), 7.40-7.28 (m, 4H), 7.08-7.05 (t, 1H), 6.86-6.85 (d, 1H), 6.36-6.33 (d, 1H), 5.71-5.69 (d, 1H), 3.87-3.72 (q, 2H), 2.60-2.57 (t, 4H), 1.91-1.88 (m, 2H), 1.75 (s, 6H), 1.66 (s, 6H), 1.36-1.33 (t, 3H).

### 10. Synthesis of Compound II-11

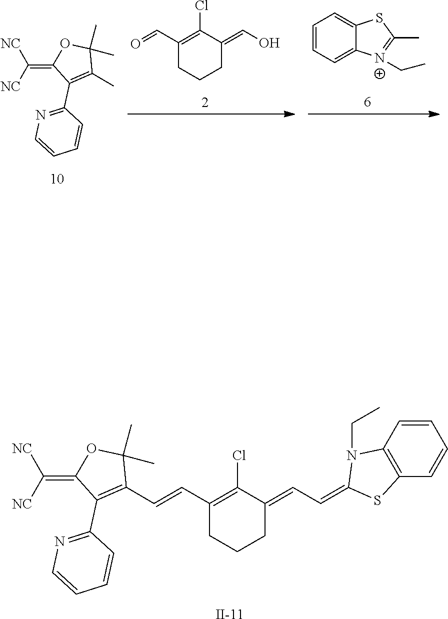

Compound II-11 was prepared by using Compound 10 and Compound 6 to replace Compound 1 and Compound 4 in Example 1, respectively, and the remaining required reagents and preparation methods used were the same as step 4) in Example 1.

¹H NMR (400 MHz, CDCl₃): δ (ppm): 8.75-8.71 (m, 1H), 8.13-8.10 (d, 1H), 7.98-7.96 (d, 1H), 7.85-7.76 (m, 1H), 7.40-7.28 (m, 4H), 7.08-7.05 (t, 1H), 6.86-6.85 (d, 1H), 6.36-6.33 (d, 1H), 5.71-5.69 (d, 1H), 3.87-3.72 (q, 2H), 2.60-2.57 (t, 4H), 1.91-1.88 (m, 2H), 1.75 (s, 6H), 1.36-1.33 (t, 3H).

### 11. Synthesis of Compound II-12

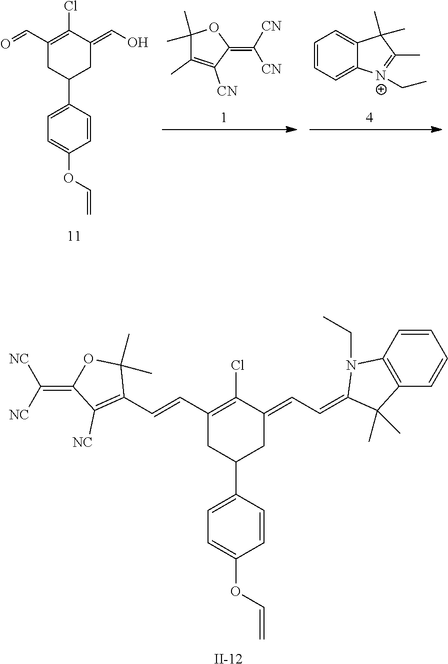

Compound II-12 was prepared by using Compound 11 to replace Compound 2 in Example 1, and the remaining required reagents and preparation methods used were the same as step 4) in Example 1.

¹H NMR (400 MHz, CDCl₃): δ (ppm): 7.34 (d, 1H), 7.26 (d, 2H), 7.06 (d, 1H), 6.79 (d, 4H), 6.51 (m, 4H), 5.41 (m, 2H), 5.01 (t, 1H), 4.67 (d, 2H), 4.13 (q, 2H), 2.84 (t, 1H), 2.36 (d, 4H), 1.79 (t, 3H), 1.66 (s, 6H), 1.35 (s, 6H).

### 12. Synthesis of Compound II-13

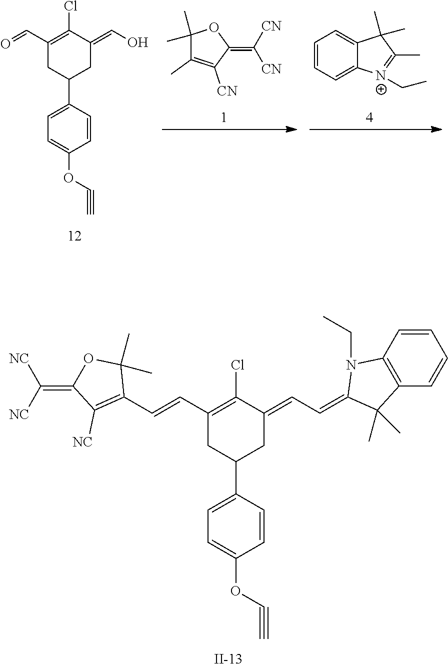

Compound II-13 was prepared by using Compound 12 to replace Compound 2 in Example 1, and the remaining required reagents and preparation methods used were the same as step 4) in Example 1.

¹H NMR (400 MHz, CDCl₃): δ (ppm): 7.34 (d, 1H), 7.26 (d, 2H), 7.06 (d, 1H), 6.79 (d, 4H), 6.51 (m, 4H), 5.41 (m, 2H), 5.01 (t, 1H), 4.67 (d, 1H), 4.13 (q, 1H), 2.84 (t, 1H), 2.36 (d, 4H), 1.79 (t, 3H), 1.66 (s, 6H), 1.35 (s, 6H).

### 13. Synthesis of Compound II-14

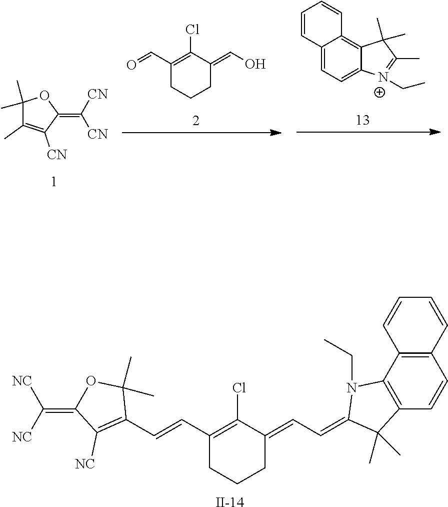

Compound II-14 was prepared by using Compound 13 to replace Compound 4 in Example 1, and the remaining required reagents and preparation methods used were the same as step 4) in Example 1.

¹H NMR (400 MHz, CDCl₃): δ (ppm): 7.34 (d, 2H), 7.26 (d, 2H), 7.06 (d, 2H), 6.79 (d, 4H), 6.51 (m, 4H), 5.41 (m, 2H), 5.01 (t, 1H), 4.67 (d, 2H), 4.13 (q, 2H), 2.84 (t, 1H), 2.36 (d, 4H), 1.79 (t, 3H), 1.66 (s, 6H), 1.35 (s, 6H).

### 14. Synthesis of Compound II-15

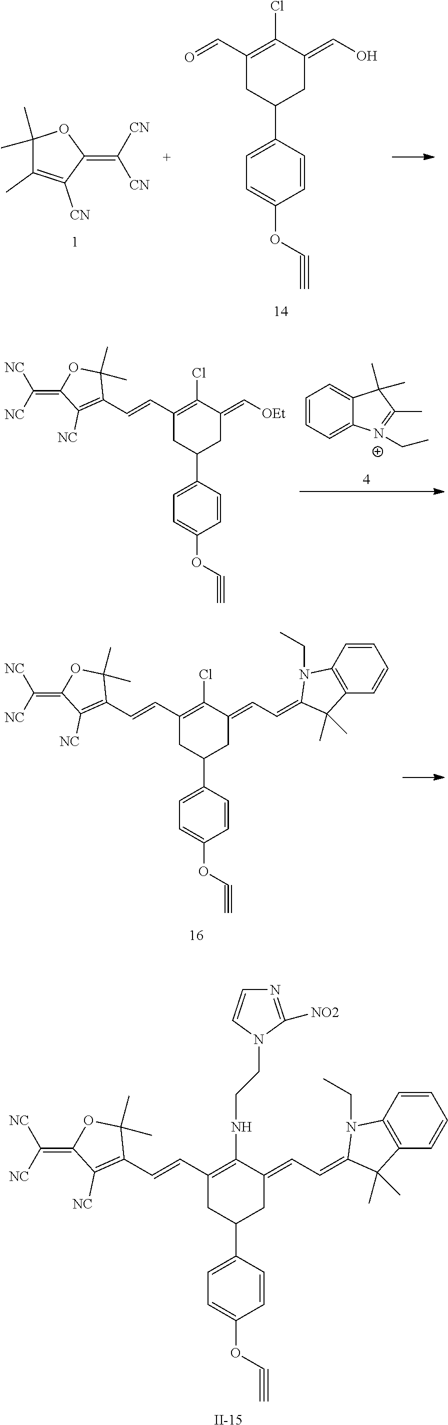

Compound 16 was prepared by using Compound 14 to replace Compound 2 in Example 1, and the remaining required reagents and preparation methods used were the same as step 4) in Example 1. Compound 16 and nitroimidazole derivative were stirred in DMF solvent at 55° C. for 24 hours, the solvent was removed by evaporation under vacuum, and the resulting solid was purified by column chromatography to obtain the target Compound II-15.

¹H NMR (400 MHz, CDCl₃): δ (ppm): 8.33 (d, 2H), 7.34 (d, 2H), 7.26 (d, 2H), 7.06 (d, 2H), 6.79 (d, 4H), 6.51 (m, 4H), 5.41 (m, 2H), 5.01 (t, 1H), 4.54 (t, 2H), 4.67 (d, 2H), 4.13 (q, 2H), 3.61 (t, 2H), 2.84 (t, 1H), 2.36 (d, 4H), 1.79 (t, 3H), 1.66 (s, 6H), 1.35 (s, 6H).

### 15. Synthesis of Compound II-16

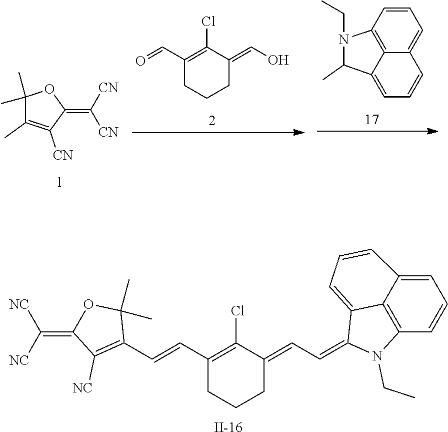

Compound II-16 was prepared by using Compound 17 to replace Compound 4 in Example 1, and the remaining required reagents and preparation methods used were the same as step 4) in Example 1.

¹H NMR (400 MHz, CDCl₃): δ (ppm): 7.96 (d, 1H), 7.82 (d, 1H), 7.64-7.60 (t, 2H), 7.45 (t, 1H), 7.04 (d, 1H), 6.51 (d, 2H), 6.23 (d, 2H), 4.15-4.10 (m, 2H), 2.82-2.79 (t, 4H), 1.47 (m, 2H), 1.31 (t, 3H), 1.16 (s, 6H).

### 16. Synthesis of Compound II-17

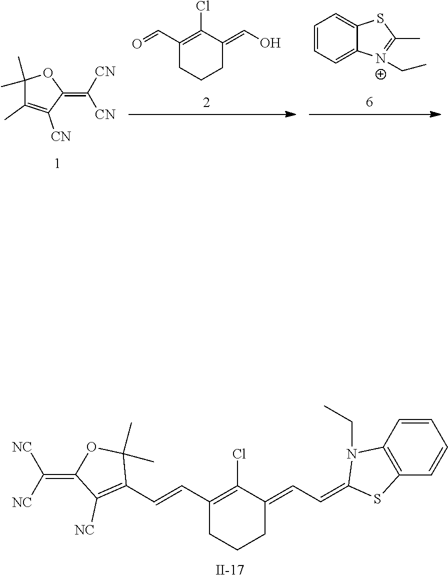

Compound II-17 was prepared by using Compound 6 to replace Compound 4 in Example 1, and the remaining required reagents and preparation methods used were the same as step 4) in Example 1.

¹H NMR (400 MHz, CDCl₃): δ (ppm): 7.88 (d, 1H), 7.56 (d, 1H), 7.48 (t, 1H), 7.29 (t, 1H), 6.51 (d, 2H), 6.23-6.17 (d, 2H), 4.38 (m, 2H), 2.81-2.79 (m, 4H), 1.47 (m, 2H), 1.40 (t, 3H), 1.16 (s, 6H).

### 17. Synthesis of Compound II-18

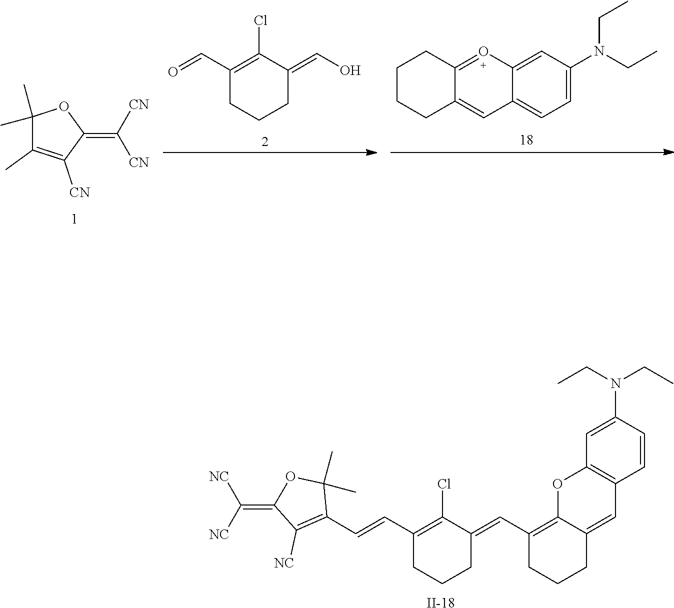

Compound II-18 was prepared by using Compound 18 to replace Compound 4 in Example 1, the remaining required reagents and preparation methods used were the same as step 4) in Example 1.

¹H NMR (400 MHz, CDCl₃): δ (ppm): 6.99 (d, 1H), 6.63 (d, 1H), 6.51 (d, 2H), 6.33 (d, 1H), 6.23 (s, 1H), 6.17 (s, 1H), 3.36 (m, 4H), 2.84-2.77 (m, 8H), 1.49-1.43 (m, 4H), 1.16 (s, 6H), 1.12-1.10 (t, 6H).

### Example 3 Ultraviolet Spectrum Characterization of Compounds II-16 to II-18

Compounds II-16 to II-18 were prepared as a storage solution with a concentration of 2 mM. The ultraviolet absorption spectra of Compounds II-16 to II-18 in water and acetonitrile were shown in FIG. 4. It can be seen that the absorption spectra of Compound II-16 in the two solvents differed greatly in intensity, but the shape of the absorption spectra was similar. The shape of the absorption spectra of Compound II-17 in the two solvents was significantly different. The absorption spectrum in water was flat, and showed a blue-shifted peak of H aggregation, but the ultraviolet absorption spectrum in the organic solvent acetonitrile showed a sharp red-shifted peak. The UV absorption spectra of II-17 in the two solvents not only had a large difference in absorption intensity, but also had a large difference in the shape of the absorption spectra. The UV absorption spectra of II-18 molecule in water and that in organic solvent were very different. There is an obvious J aggregation absorption peak in organic solvent, but there was an obvious H aggregation peak in water, and the absorbance in water was significantly lower. The results show that these three molecules II-16, II-17 and II-18 have their own unique assembly methods in water.

### Example 4 Preparation of Micro-Nano Structure

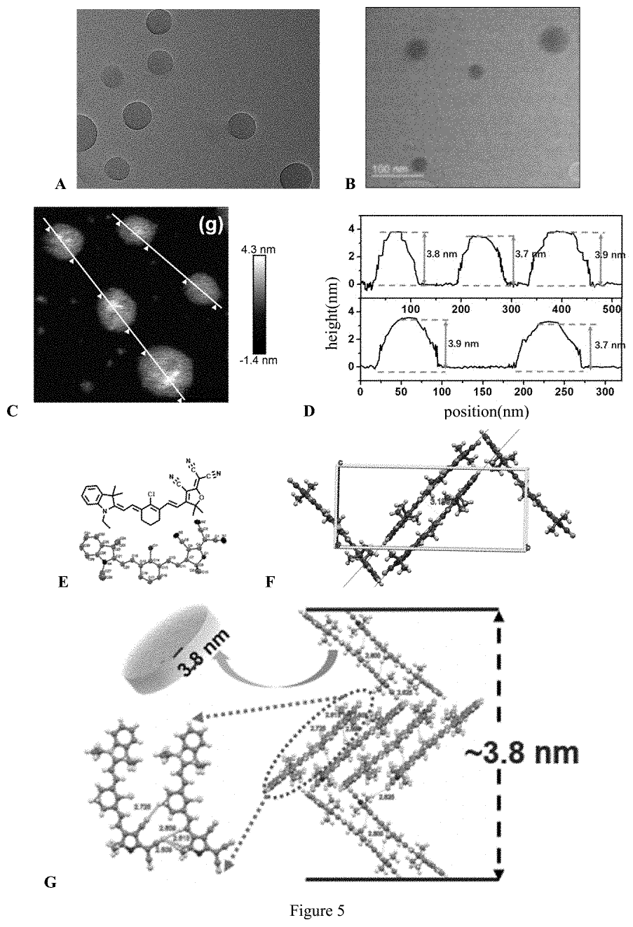

Taking the self-assembled micro-nano structure of Compound II-1 as an example, II-1 was dissolved in DMSO (also can be an organic solvent such as ethanol) to prepare a 2 mM storage solution, and a small amount of storage solution was added to deionized water to prepare 20 μM working solution. Took 10 μL and dropped it on copper mesh, observed and analyzed under transmission electron microscope (TEM), cryo-transmission electron microscope. Took 10 μL and dropped it on mica plate, observed and analyzed under atomic force microscope (AFM). Photographs and data were shown in FIG. 5. In FIGS. 5, A, B, C and D were TEM photographs, cryo-transmission electron microscope photographs, AFM photographs, and AFM analysis data, respectively. According to the photographs and data, II-1 was self-assembled into nanosheets with a diameter of 40-70 nm and a thickness of 3.7-3.9 nm.

At the same time, the crystal structure of II-1 was analyzed. The crystal structure of II-1 shows that each unit cell consists of 4 II-1 molecules, and the unit cell has a length of 3.8 nm. Countless unit cells were stacked by Diamond software to build a nanosheet with a diameter of 60 nm and a thickness of 3.8 nm to simulate the nanosheet structure of II-1. The molecular structure of II-1 and its corresponding crystal structure, unit cell structure of II-1, and the structure of the nanosheet simulated by Diamond software were shown in E, F and G of FIG. 5, respectively.

### Example 5 Charged Cyanine Compounds are Similar to the Structure of the Compound of the Present Invention, but they Cannot be Self-Assembled to Form a Micro-Nano Structure

Four kinds of charged cyanine compounds ICG, Cy-1, Cy-2, and Cy-3 were prepared in DMSO as 2 mM storage solutions, and then a small amount of storage solution was added to deionized water to prepare a 20 μM working solution. The particle diameter was tested by DLS, it was found that the particle size of the four cyanine compounds ICG, Cy-1, Cy-2, and Cy-3 could not be detected, indicating that they could not form a micro-nano structure in water.

### Example 6 Comparison of Characteristic Data of Micro-Nano Structures Formed by Self-Assembly of Compounds of the Present Invention

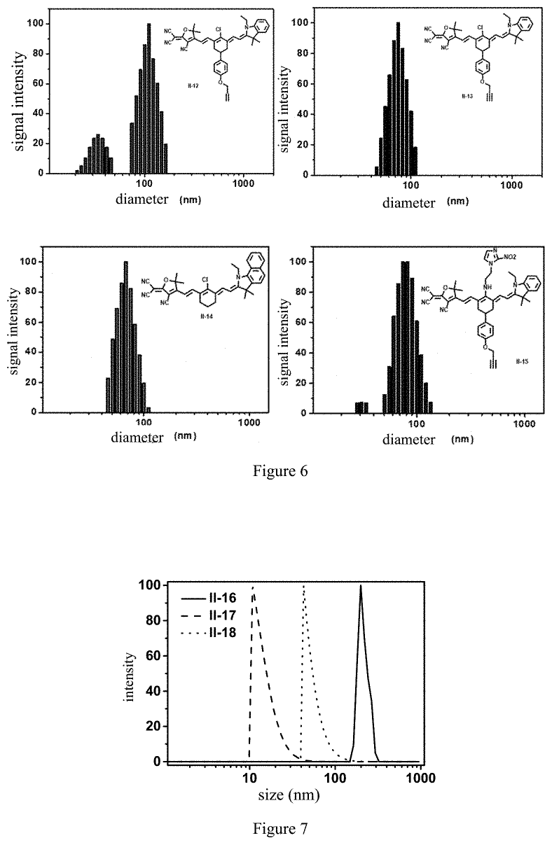

Compounds II-1, II-12 to II-18 were treated by the method of Example 4, and it was found that all of Compounds II-1 and II-12 to II-18 showed self-assembly behavior in aqueous solution, and assembled to form a micro-nano structure, the comparison of the characteristics data is shown in Table 2. Among them, the particle size test results of II-12 to II-15 in aqueous solution are shown in FIG. 6, and the results of dynamic light scattering DLS of II-16 to 1-18 are shown in FIG. 7).

TABLE 2
Characteristic data of the micro-nano structure
formed by the compound of the present invention

| Compound | DLS particle | DLS particle | |
|---|---|---|---|
| number | size range (nm) | size peak (nm) | Morphology |
| II-1 | 33-266 | 104 | nano-sheet |
| II-12 | 20-160 | 78 | nano-sheet |
| II-13 | 40-110 | 74 | nano-sheet |
| II-14 | 27-134 | 78 | nano-sheet |
| II-15 | 40-110 | 60 | nano-sheet |
| II-16 | 163-293 | 199 | nano-sheet |
| II-17 | 10-86 | 10 | nano-sheet |
| II-18 | 42-139 | 42 | nano-sheet |

It can be seen from the above table that the particle size of the Compound II-1, II-12 to II-18 all can be measured, and the particle size range is within the range of 10-300 nm, which belongs to the micro-nano structure. According to the morphology and data characteristics of AFM and TEM, it can be found that Compounds II-1, II-12 to II-18 all formed nanosheet structures.

The examples of other compounds of the present invention were tested and found that all of the compounds of the present invention can form a micro-nano structure in an aqueous solution, but none of the four cyanine compounds described in Reference Example 5 can form a micro-nano structure in an aqueous solution. It shows that the uncharged compounds are important for the formation of micro-nano structures.

### Example 7 Calculation of Quantum Yield of the Compound of the Present Invention

The Compound II-1 of the present invention was prepared as a 2 mM storage solution in DMSO, and then 10 μL was added to 2 mL of solutions with different polarities (including water, dimethyl sulfoxide, N,N-dimethylformamide, methanol, ethanol, acetone, methylene chloride, acetonitrile), a UV spectrophotometer was used to test the UV absorption and to find the maximum absorption wavelength. The molar extinction coefficient of II-1 in solutions of different polarities was calculated from the ultraviolet absorption spectrum. Excited by the maximum absorption wavelength, the fluorescence emission spectrum was tested by a fluorescence spectrometer, the data was plotted with origin and the integral area was calculated. The fluorescence quantum yield φₓ, φₓ=φs(Fₓ/Fₛ)(Aₛ/Aₓ)(λₑₓₛ/λₑₓₓ)(nₓ/nₛ)², was calculated from the obtained data to obtain the characteristic parameters of Compound II-1 in solutions of different polarities (Table 3).

TABLE 3
Characteristic parameters of Compound II-1
in solutions of different polarities

| | λₘₐₓ/ | λₑₘ/ | ε*10⁴/ | dielectric | |
|---|---|---|---|---|---|
| | nm | nm | M⁻¹cm⁻¹ | constant | Φ/% |
| water | 730 | 840 | 2.76 | 80.4 | 0.11 |
| dimethyl sulfoxide | 848 | 854 | 10.24 | 47.2 | 0.12 |
| N,N-dimethylformamide | 844 | 853 | 7.92 | 37.6 | 0.19 |
| methanol | 830 | 850 | 11.24 | 33.6 | 0.19 |
| ethanol | 828 | 851 | 10.74 | 24.3 | 0.25 |
| acetone | 832 | 851 | 8.91 | 20.7 | 0.29 |
| dichloromethane | 834 | 850 | 8.55 | 8.9 | 0.35 |
| acetonitrile | 832 | 850 | 9.15 | 38.8 | 0.35 |

It can be seen from the above table that the characteristic parameters of Compound II-1 in solutions of different polarities are different, especially the maximum absorption wavelength and maximum excitation wavelength in water are significantly different from those in other organic solvents, and their quantum yield are significantly reduced, and the photothermal effect is stronger. The essence of this phenomenon is that Compound II-1 self-assembles in aqueous solution to form a micro-nano structure. Due to changes in structural properties, its physical properties and characteristic parameters change, and this change is conducive to improving the photothermal effect and light stability. In fact, not only Compound II-1 has such characteristics, other compounds of the present invention also have similar properties, and can self-assemble in aqueous solutions to form micro-nano structures.

### Example 8 Photothermal Effect and Photothermal Stability of Compound II-1 In Vitro

A total of 4 groups of samples, 3 mL was taken and added to a cuvette respectively, and the lid was sealed.

The sample No. 1 was 3 mL deionized water;

The sample No. 2 was 10 μM II-1, and the specific preparation method is 3 mL deionized water plus 15 μL II-1 stock solution (2 mM, dissolved in DMSO);

The sample No. 3 was 20 μM II-1, and the specific preparation method was that 3 mL deionized water was added 30 μL II-1 stock solution (2 mM, dissolved in DMSO);

The sample No. 4 was 40 μM II-1, and the specific preparation method was that 3 mL deionized water was added 60 μL II-1 stock solution (2 mM, dissolved in DMSO).

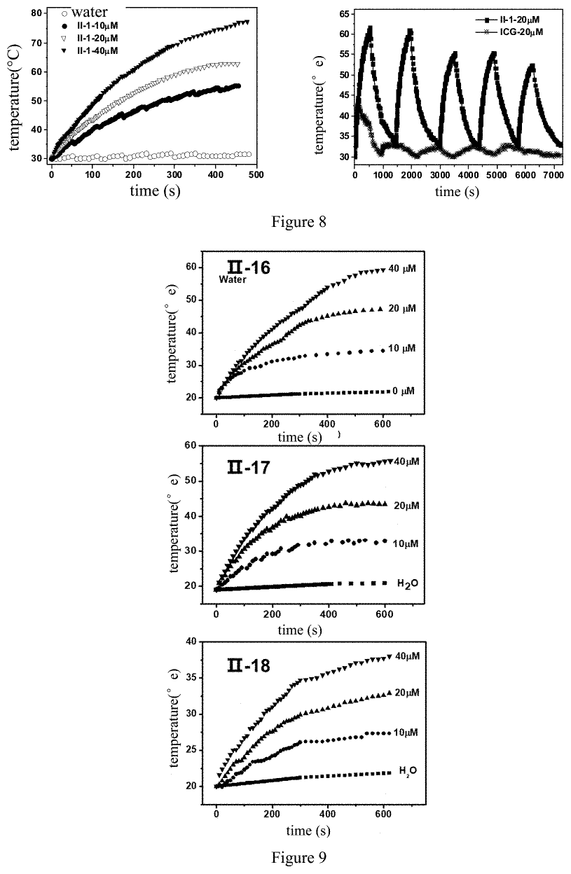

Each sample was irradiated with an 808 nm laser for 5 minutes, and the temperature data was recorded every 5 seconds with a thermal imager, and the temperature corresponding to the time was plotted in origin, as shown in the left figure of FIG. 8. The temperature of sample No. 1 was almost unchanged within 5 minutes, and only increased by 2° C. The temperature of sample No. 2 increased from room temperature 30° C. to 54° C., and the temperature was raised by 24° C. The temperature of sample No. 3 increased from room temperature 30° C. to 62° C., and the temperature was raised by 32° C. The temperature of sample No. 4 increased from room temperature 30° C. to 77° C., and the temperature was raised by 47° C. It shows that Compound II-1 has excellent photothermal effect.

The light-to-heat conversion efficiency of samples No. 2, No. 3 and No. 4 were calculated, and found that the light-to-heat conversion efficiency of sample No. 2 was 60.4%, the light-to-heat conversion efficiency of sample No. 3 was 61%, and the light-to-heat conversion efficiency of sample No. 4 was 60%. It also shows that Compound II-1 has extremely excellent photothermal effect.

We selected sample No. 3 to test the photothermal stability experiment. As shown in the right figure of FIG. 8, sample No. 3 was irradiated with 808 nm laser for 9 minutes and then the temperature increased from room temperature 30° C. to 62° C., and then allowed to naturally cool down to room temperature, and irradiated with 808 nm laser again for 9 minutes, then naturally cooled down, and repeated for 5 times. It was found that in these 5 repeated experiments, sample No. 3 could be raised from room temperature to at least 55° C. every time under the irradiation of 808 nm laser, and the temperature was raised by 25° C. ICG with a similar structure, which has been commercialized, does not have this characteristic. After 9 minutes of irradiation, the temperature was raised by 15° C., and then returned to room temperature. After laser irradiation again, the temperature cannot be increased.

Therefore, the Compound II-1 of the present invention not only has an excellent photothermal effect, but also has excellent photothermal stability that other organic small molecule fluorescent compounds do not have, which overcomes the defects of the organic small molecule compound in terms of photothermal stability and has potential prospects for clinical application. Other compounds of the invention also have similar properties.

### Example 9 Photothermal Effect and Photothermal Stability of Compound II-16 to II-18 In Vitro

Using a method similar to Example 8, 10 μM, 20 μM and 40 μM samples of II-16, II-17 and II-18 were prepared, respectively, they were irradiated under an 808 nm (2.6 W/cm-2) laser for 10 minutes. The elevated temperature of the three samples with different concentrations in water was shown in FIG. 9. 40 μM II-16 to 18 samples can rise by 40° C., 36° C. and 18° C. respectively within 10 minutes under laser irradiation. The results show that II-16 and II-17 have excellent photothermal effect, while II-18 has poor photothermal effect.

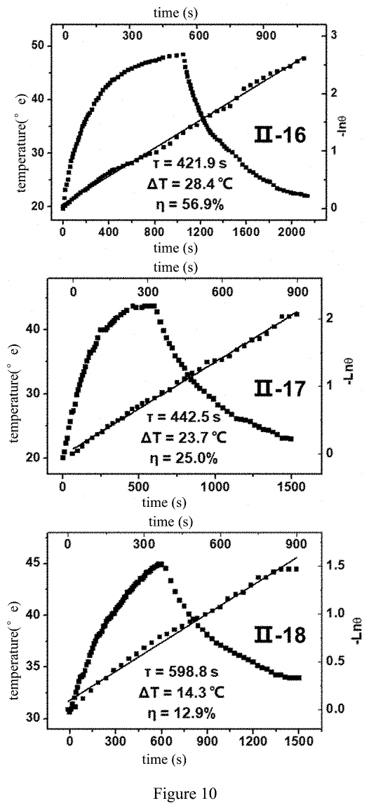

Using a method similar to that in Example 8, three samples of 20 μM of 1-16, II-17 and II-18 in water were irradiated with 808 nm (2.6 W/cm-2) laser for 10 minutes, and the light-to-heat conversion efficiency calculation result thereof were shown in FIG. 10. II-16 not only has an excellent light-to-heat conversion effect, and its light-to-heat conversion efficiency is as high as 56.9%, while II-17 is excellent in photothermal effect and is comparable to II-16, but its light-to-heat conversion efficiency is only 25%. And II-18 not only has poor light-heat effect, but also has low light-heat conversion efficiency, only 12.9%.

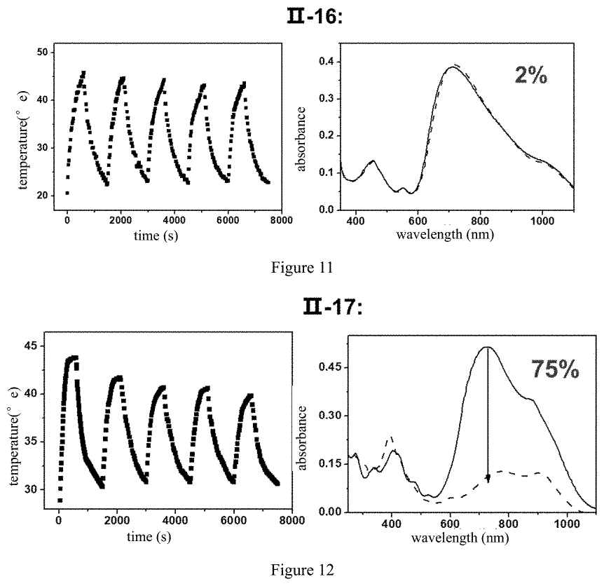

In view of the poor photothermal effect and light-to-heat conversion efficiency of II-18, only the photothermal stability of II-16 and II-17 were tested. As shown in FIG. 11, the photothermal stability of II-16 is very good. 808 nm laser at 2.6 W/cm-2 repeatedly irradiated and cooled for 5 times, each time the temperature can rise to the same temperature as the first time (left in FIG. 11), and it can be seen from the ultraviolet spectra of the sample before and after 5 times of laser irradiation (right in FIG. 11) that the absorbance of the sample after 5 times of irradiation is reduced by only 2%, indicating that the sample has very good photothermal stability. The photothermal stability of II-17 is ordinary (FIG. 12), especially from the ultraviolet spectra before and after laser irradiation (right in FIG. 12), the absorbance of the sample is reduced by 75%, indicating that the photothermal stability of II-17 is poor. In terms of photothermal effect, light-to-heat conversion efficiency and photothermal stability, II-16 has excellent photothermal effect, high light-to-heat conversion efficiency and excellent photothermal stability, and has good application prospects.

### Example 10 Assembly Stability of Compound II-16 in Different Media

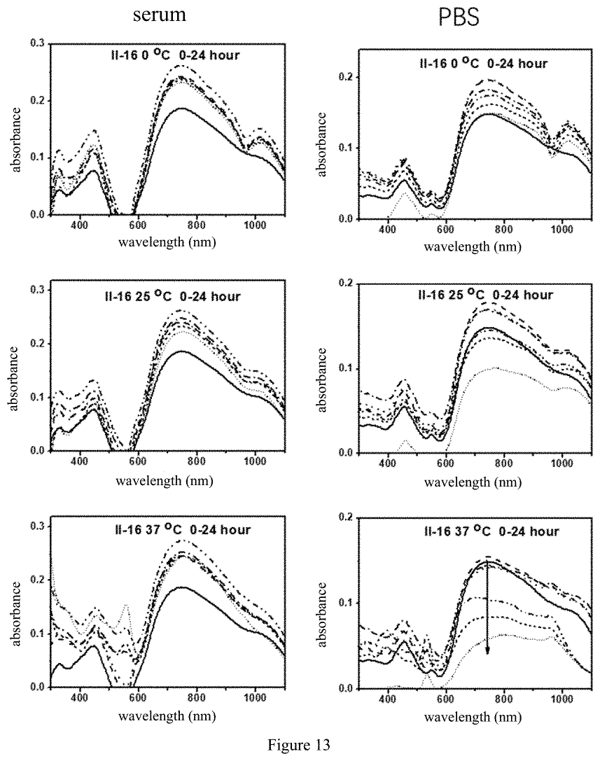

Since II-16 has excellent photothermal properties, its assembly stability in serum-containing medium and in PBS at different temperatures was tested, as shown in FIG. 13. The aggregation mode of II-16 (H aggregation) will not change due to changes in external conditions. At 37° C., the absorbance will decrease with time which does not affect its own aggregation mode.

### Example 11 Cell Photothermal Experiment of Compound II-1

HeLa cells were digested from the culture flask with trypsin, centrifuged, added to a DMEM medium containing 10% serum and 1% double antibody, and mixed well. 20 μL of cells were taken and stained with Compound II-1 (20 μM) for 30 minutes. After taking it out, it was centrifuged and rinsed twice with PBS. After centrifuging, 20 μL of culture medium and 20 μL of trypan blue were added. 20 μL was taken from the mixture to the cell count plate and photographed under fluorescence microscope. In the absence of laser irradiation, bright live cells were observed under the microscope, and the cell survival rate was 92%. When irradiated with 808 nm laser for 6 minutes, almost all the blue dead cells were observed under the microscope, and the cell death rate was as high as 100%. HeLa cells were cultured in 96-well plates, 10⁴ cells per well, and live/dead staining after 24 h. The cells were irradiated with an 808 nm laser for 6 minutes, and the photographs were observed under a fluorescent microscope. The cells were all red, indicating that the cells were 100% dead, while the cells were all green in the control group (only the compound was added, without laser irradiation) under the fluorescent microscope, the cells were all alive. It can be seen that the toxicity of the compound itself is extremely small, but its photothermal effect is particularly lethal to cancer cells, and it has bright prospects in the future clinical application of photothermal treatment of cancer. Other compounds of the present invention also have similar photothermal therapeutic effects.

### Example 12 Cell Imaging Experiments of Compound II-1

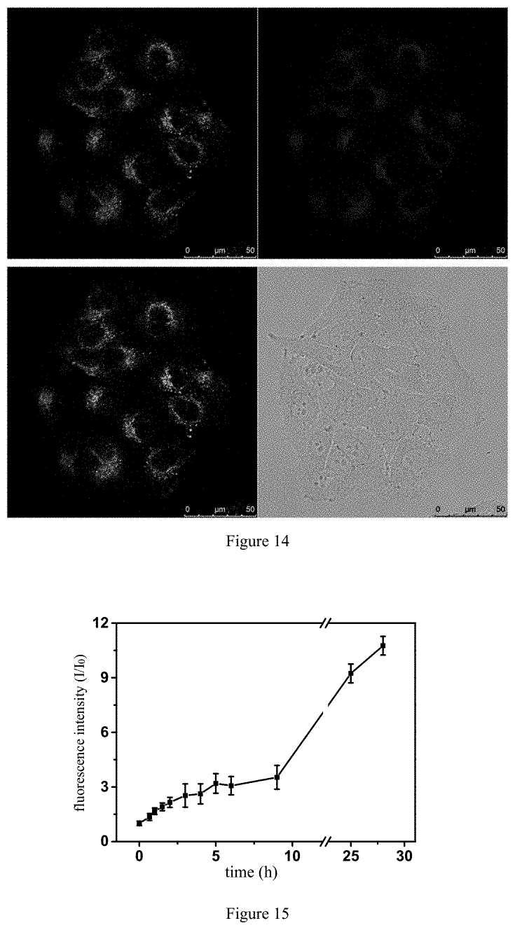

The lysosomal dyes Lyso-Green (75 nM) and II-1 (8 μM) were added to the cell culture medium for 30 minutes of cell staining. After dyeing, the cells were washed twice with PBS and observed and photographed under a confocal fluorescence microscope. Combining the photos of the green channel and the red channel together, it is found that the cells were yellow without no obvious red and green color, as shown in FIG. 14. It shows that the red channel and the green channel are almost completely coincident, indicating that Compound II-1 is a dye that can target the lysosome.

### Example 13 Fluorescence Imaging, Photothermal Imaging, Photothermal Therapy and Photoacoustic Imaging of Compound II-1 on Mice in the Subcutaneous Metastasis Model

1. Establishment of Model

The left side of 6-week-old female nude mice was injected subcutaneously with 107 4T1 cells to grow the tumor volume to 60 mm³.

2. Fluorescent Imaging

In the experimental group, 200 μL of II-1 (1 mg/mL) was injected into the tail vein of mice. After injection, the living imager was used to monitor at different times. As shown in FIG. 15, it was found that the fluorescence intensity of the tumor site was gradually increased over time. After 24 hours, there was no compound in other parts of the body, all enriched in the tumor site (FIG. 16). Then they were dissected, and the heart, liver, spleen, lung, kidney and tumor were imaged by fluorescence. It was found that the fluorescence was very strong at tumor site, with weak fluorescence in the liver, and no fluorescence in other parts. This proves that Compound II-1 targets the tumor site very well. Moreover, within 24 hours, no abnormalities such as spasms and convulsions occurred in the body of nude mice, which proved that Compound II-1 was almost non-toxic and extremely safe.

3. Photothermal Imaging

In the experimental group, 200 μL of II-1 (300 μmoL) was injected into the mouse through the tail vein, and the tumor site of the mouse was irradiated with an 808 nm laser for 10 minutes, while taking photos with a photothermal imaging instrument. Under laser irradiation, the tumor site can be heated to 60° C. As can be seen from FIG. 17, the temperature of the tissue around the tumor has not been increased, indicating that II-1 used in photothermal therapy has the advantage of low damage to the tissue near the tumor.

4. Photothermal Treatment In Vivo

The nude mice were divided into 4 groups. In the first group, 200 μL of II-1 was injected into the mouse through the tail vein, and the tumor site of the mouse was irradiated with an 808 nm laser for 5 minutes. In the second group, 200 μL of II-1 (300 μmol) was injected, no laser irradiation. In the third group, saline was injected and laser irradiated for 5 minutes. In the fourth group, saline was injected, no laser irradiation. The tumor volume of each group of mice was measured daily with a vernier caliper, and the recording was continued for 30 days.

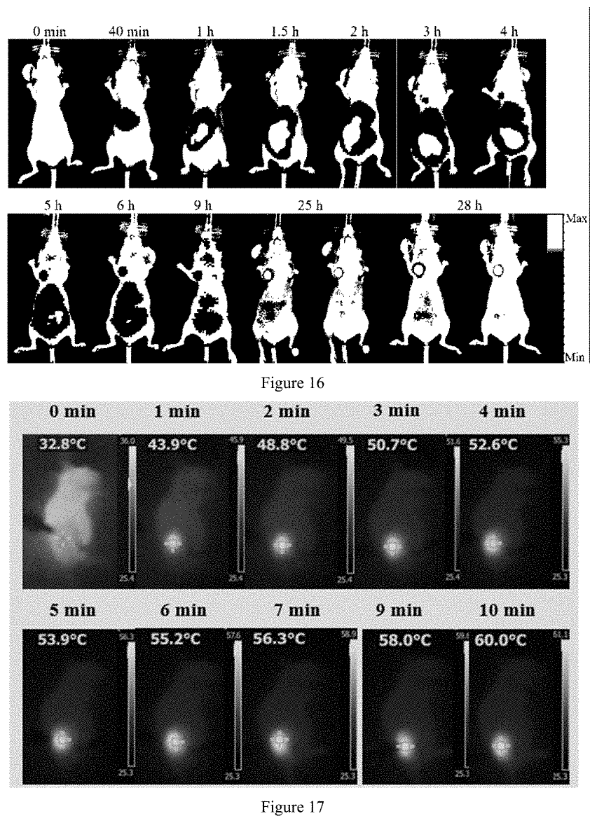

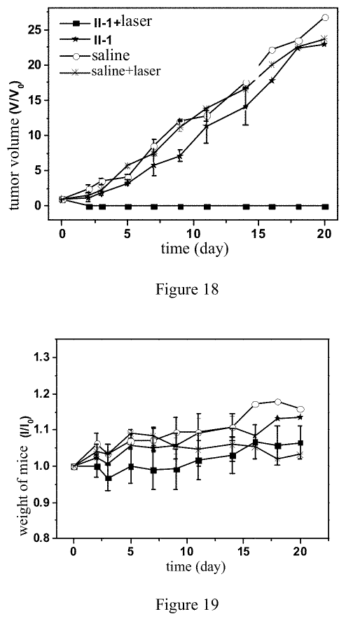

As shown in FIG. 18, the nude mice after photothermal treatment in the first group had a tumor volume of about 70 mm³ before photothermal treatment. After photothermal treatment, the tumors ruptured the next day. There was no obvious tumor growth was seen with time. The tumor ulcer began to heal, and the ulcer had completely healed on day 16, with a small scar. The change in tumor volume was shown in FIG. 18. In the experimental group (Group 1), the tumor disappeared after laser irradiation, and there was no recurrence within 20 days. In contrast, the tumor volume of mice in the control group (groups 2, 3 and 4) continued to rise, and had grown 25 times in 20 days. The weight change of mice was shown in FIG. 19. There is no abnormal change in the body weight of the experimental group and the control group, and no obvious side effects of II-1 have been seen. The mice in the experimental group 20 days after the experiment were dissected, and the tumor, liver and lung were sliced and observed by H&E staining. It was found that the tumor cells had undergone apoptosis, and there was no obvious damage to the lungs and liver. It proves that II-1 has excellent photothermal treatment ability, and is relatively safe and reliable without damage to internal organs and side effects.

The above experiment shows that Compound II-1 has very good photothermal killing effect on tumors under 808 nm laser irradiation, and has high safety, and has broad application prospects in clinical photothermal treatment of cancer. It has been verified that other compounds of the present invention have similar photothermal treatment effects.

5. Photoacoustic Imaging

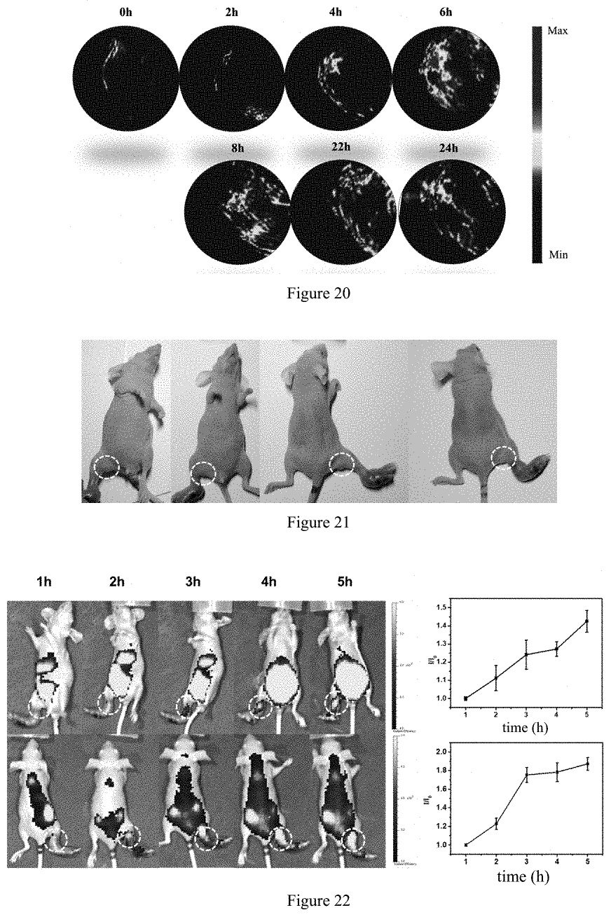

200 μL II-1 (300 μmoL) was injected into the mice of the experimental group through the tail vein. Monitored with multispectral photoacoustic tomography at different times. As shown in FIG. 20, no signal was found within 2 hours after injection, and a clear photoacoustic signal was found at the tumor site at 4 hours. With the increase of time, until 24 hours, there was a photoacoustic signal at the tumor site. This example demonstrates that Compound II-1 has excellent tumor targeting and excellent photoacoustic signal.

### Example 14 Fluorescence Imaging and Photothermal Treatment of Compound II-1 in Sentinel Lymphatic Metastasis Model Mice

1. Establishment of Mouse Metastasis Model

Cell preparation: The cells in the culture flask were digested with trypsin and placed in a centrifuge tube for use. The supernatant was discarded by centrifugation and counted. Then a large amount of medium was added and it was placed in a foam box to be taken to the company. The supernatant was removed by centrifugation, and about 3.5 mL of serum-free medium was added. It was prepared for injection.

Establishment of nude mouse tumor model: 40 mice, 6-week-old female nude mice were injected with 106 T4 cells (25 μL) in the right hind paw.

The tumor was established as shown in FIG. 21, and obvious sentinel lymph nodes can be found in the popliteal area of the right hind leg of the mouse. After that, small animal fluorescence imaging and photothermal therapy can be performed.

2. Fluorescent Imaging

Fluorescence imaging for the small animal was performed on the established mouse metastasis model. Using tail vein injection and intratumoral injection (FIG. 22), fluorescence detection was performed every 1 hour for a total of 5 hours. By comparison, it was found that intratumoral injection of II-1 can obviously enter the sentinel lymph node of mice, and the effect is more excellent than that of tail vein injection.

3. Photothermal Treatment

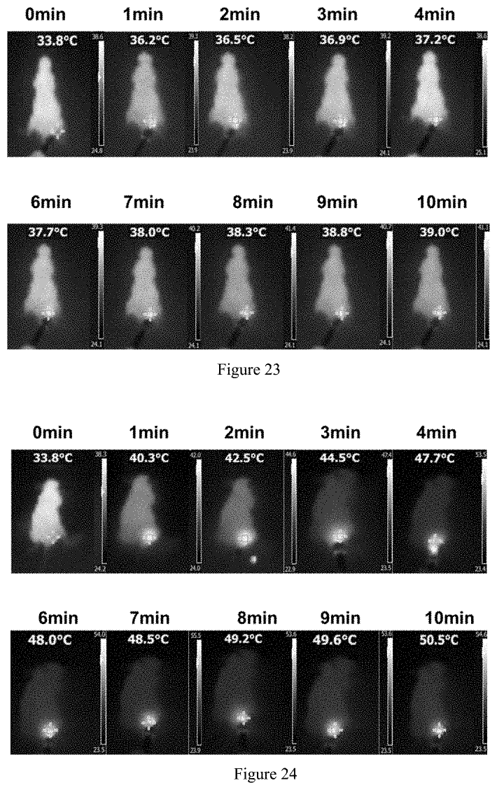

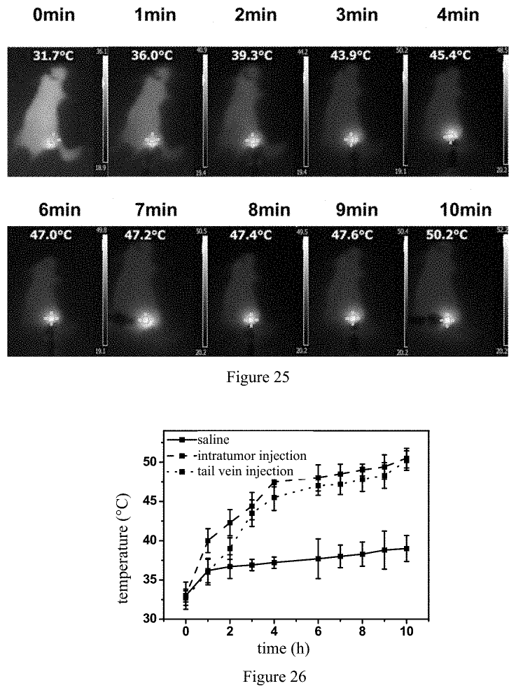

The mice were treated with photothermal therapy after injection of tumor cells on the right hind foot pad with obvious sentinel lymph nodes (about 20 days). In this experiment, mice were randomly divided into 3 groups, each group 5 mice, saline+laser irradiation control group, intratumoral injection+laser irradiation experimental group and tail vein injection+laser irradiation experimental group, respectively. The data of the sentinel lymph nodes of each group mice irradiated with 808 nm laser (1.5 W/cm2) for 10 minutes can be seen in FIGS. 23-25. According to the following figures, it can be found that under the 808 nm laser irradiation, the temperature in the intratumoral injection and tail vein injection group is significantly higher than that in the saline injection group, which can play a better role in killing the tumors in the sentinel lymph nodes. FIG. 26 is a clear comparison of the temperature changes at the sentinel lymph nodes after laser irradiation in the three groups.

4. Taking Photos after Photothermal Therapy

Ink dye: (15%) India Ink

Fekete's solution: 100 mL 70% ethanol+5 mL acetic acid+10 mL formaldehyde

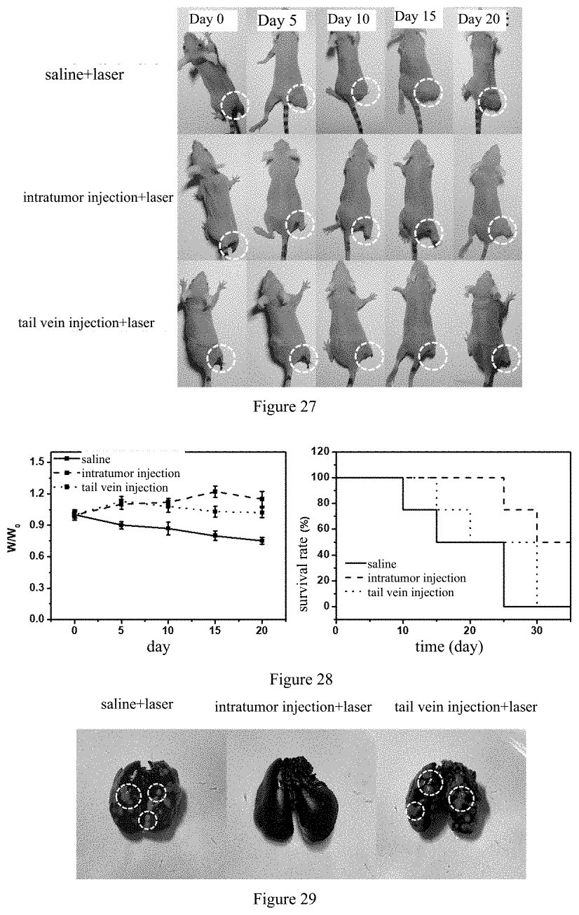

After photothermal treatment of the mice, the mice were photographed and their weights were recorded every 5 days. According to FIG. 27, it can be found that, after laser irradiation, the experimental group that injected drugs into the tumor and injected drugs into the tail vein had obvious scars at the sentinel lymph nodes of the mice. The saline group did not have similar scars because of the lower temperature rise. With the development of time (FIG. 27), the scars at the sentinel lymph nodes of the mice in the intratumoral injection group obviously healed. The scars at the sentinel lymph nodes in the tail vein injection group showed a tendency to heal, but the effect was worse than that in the intratumoral injection group. The sentinel lymph nodes in the saline injection group gradually increased. The body weight of the mice was monitored during the period (FIG. 28), it could be found that the body weight of the mice injected intratumorally had been stable in a healthy state, the weight of mice in the tail vein injection group decreased on the tenth day. In the saline injection group, the body weight of the mice had been on a downward trend, and death occurred when the weight was reduced to a certain level. The survival rate of the mice could be seen in FIG. 28. The mice in the saline injection group died on the 10th day and all died on the 25th day. The mice in the tail vein injection group died at 15 days and all died at 30th day. The mice in the intratumoral injection group only died on the 25th day. After the mice were stained with ink on the lungs on day 20, the lungs of the mice were photographed. As can be seen from FIG. 29, the lungs of mice in the saline group and the tail vein injection group showed obvious tumor metastasis, while there was no obvious tumor metastasis in the lungs of the mice in the intratumoral injection group. It can be seen that photothermal therapy by intratumoral injection of drugs should be the most effective way to treat metastatic tumors in photothermal therapy.

### Example 15 Fluorescence Imaging of Compounds II-16 to II-18 in Mice

Establishment of the nude mouse model: 6-week-old female nude mice were prepared, and 10⁶ 4T1 cells (100 μL) were injected subcutaneously on the left side of the chest to grow the tumor volume to 50 mm³ before testing.

300 μL of Compound II-16 to II-18 storage solution was added to 2 mL saline and mixed well.

Tumor imaging: one nude mouse was injected with 50 μL of saline containing Compounds II-16 or II-17 or II-18 prepared above through the tail vein. After injection, it was monitored at different times with a small animal live imager. The fluorescence intensity was recorded until the probe exited the body at the end of the circulation in the body. The time of highest fluorescence intensity was record. The second nude mouse was injected with 50 μL of the above-prepared saline containing Compounds II-16 or II-17 or II-18 through the tail vein to kill the tumor-bearing mice at the highest fluorescence intensity, and the tumor and main organs (heart, liver, spleen, lungs and kidneys) were removed for research. Each organ was rinsed with PBS, observed under a confocal laser scanning microscope, and photographed. The third nude mouse was for reserve.

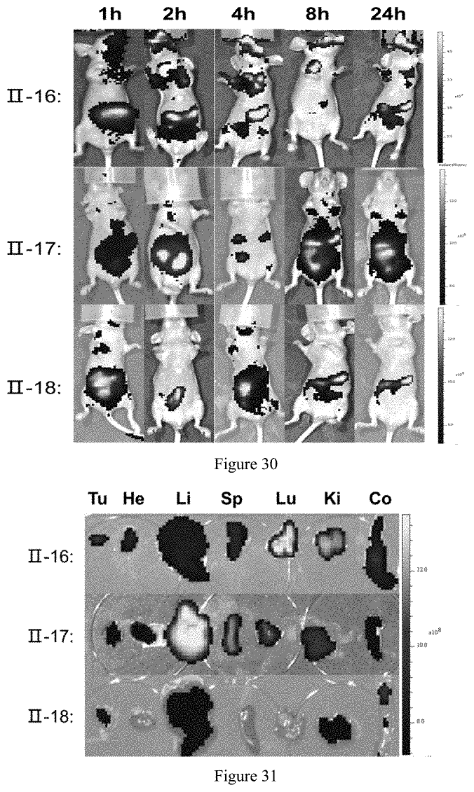

The experiment was conducted at the Medical College of Qingdao University. FIG. 30 shows the results of fluorescence imaging of Compounds II-16 to II-18 in mice. FIG. 31 is an image of internal organs of mouse.

### Example 16 Photodynamic Effect Test of Compound II-1

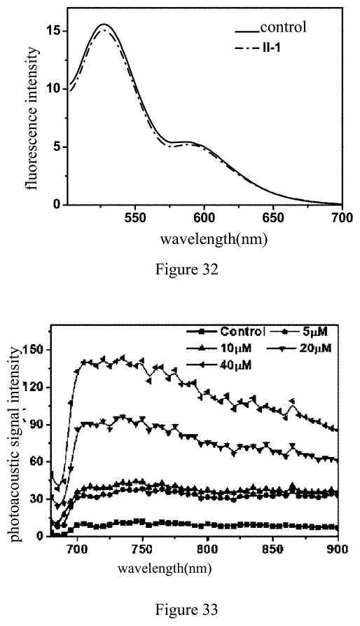

In the experimental group, carboxy-H₂DCFDA compound was added to 3 mL water, the compound had a working concentration of 25 μM, and then Compound II-1 (final concentration was 10 μM) was added. The control group only added carboxy-H₂DCFDA compound. Irradiated with 808 nm laser for 10 minutes, and the sample was lowered to room temperature, and its fluorescence emission spectrum (Ex=495 nm, Em=530 nm) was tested. As shown in FIG. 32, the fluorescence intensity of the experimental group is similar to that of the control group, indicating that Compound II-1 does not produce reactive oxygen radicals (ROS) and has no photodynamic effect. It has been verified that other compounds of the present invention have no similar photodynamic effect.

### Example 17 Photoacoustic Imaging Test of Compound II-1

In the experimental group, 2.5, 5, 10 and 20 μL of Compound II-1 (concentrations of 5, 10, 20 and 40 μM, respectively) were respectively added to 1 mL of water, and the test solution was placed in the prosthesis for photoacoustic imaging test. The test results showed that the photoacoustic signal increased with the increase of the concentration of Compound II-1, and the photoacoustic signal was the strongest at 710-750 nm (FIG. 33). This example demonstrates that Compound II-1 has a significant photoacoustic signal.

The above examples are only used to illustrate the technical solutions of the present invention, not intend to limit them. Although the present invention has been described in detail with reference to the examples, those of ordinary skill in the art can still make modifications to the technical solution recorded in the foregoing examples, or make equivalent replacement of some of its technical features; and these modifications or replacements do not deviate the essence of the corresponding technical solution from the spirit and scope of the technical solution claimed by the present invention.

1. A micro-nano structure formed by self-assembling a compound represented by formula (I), an isomer, a pharmaceutically acceptable salt, a hydrate or a solvate thereof in an aqueous solution, 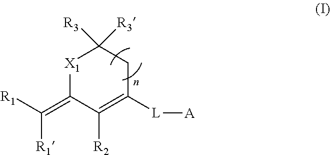
in formula (I):
A is a substituted or non-substituted heterocyclyl, which has no charge and comprises one or more heteroatoms selected from the group consisting of N, O and S;
L is a substituted or non-substituted conjugated carbon chain which comprises 2-5 double bonds;
X₁ is O, N or —CR₄R₄′—;
n is 0 or 1;
R₁, R₁′, R₂ are each independently selected from atoms and groups with electron-withdrawing ability;
R₃, R₃′, R₄ and R₄′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino;
when the group is substituted, the substituent is single or multiple.

2. The micro-nano structure according to claim 1, wherein A is 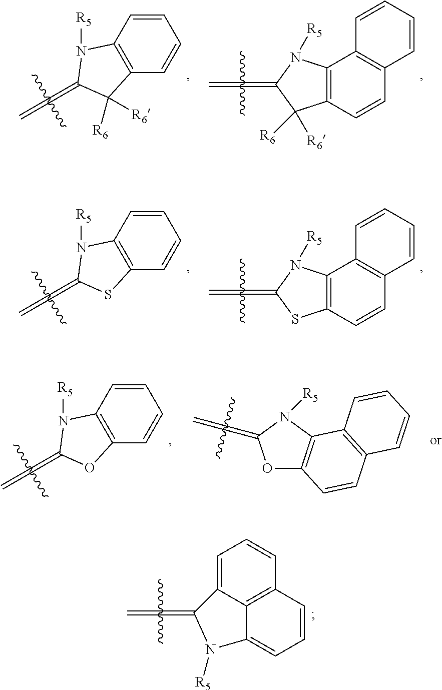
wherein R₅, R₆, R₆′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

3. The micro-nano structure according to claim 1, wherein L is 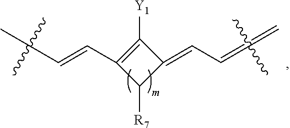
wherein Y₁ is H, halogen atom, substituted or non-substituted amino or hydrocarbyloxy;
m is an integer of 0-5;
each R₇ is independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

4. The micro-nano structure according to claim 3, wherein
m is 3;
Y₁ is Cl, Br, —NR₈R₈′ or —OR₈;
and, R₇ is H, —CH₃, 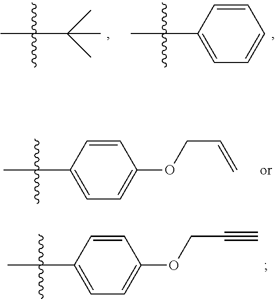
R₈ and R₈′ are each independently selected from H, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino.

5. The micro-nano structure according to claim 1, wherein R₁, R₁′, R₂ are each independently selected from —CN, —CF₃, F, —SO₂CF₃, —NO₂, —COOEt, —SO₂ph,

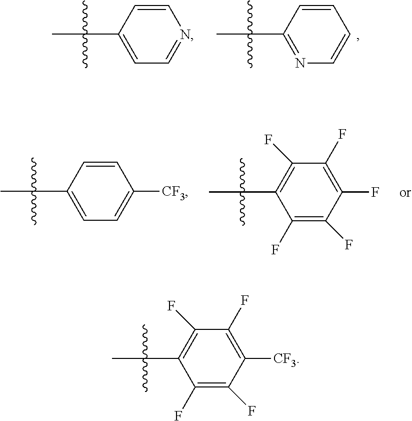 [SMILES: *c1ccncc1 ; *c1ccccn1 ; *c1ccc(C(F)(F)F)cc1 ; *c1c(F)c(F)c(F)c(F)c1F ; *c1c(F)c(F)c(C(F)(F)F)c(F)c1F]

6. A micro-nano structure formed by self-assembling a compound represented by formula (II), an isomer, a pharmaceutically acceptable salt, a hydrate or a solvate thereof in an aqueous solution, 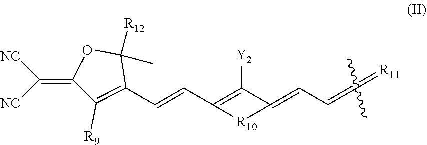
in the formula (II),
Y₂ is Cl Br, 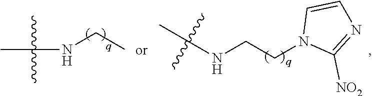
wherein q and q′ are each independently an integer selected from 0-12;
R₉ is —CN or 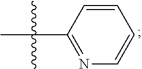 [SMILES: *c1ccccn1]
R₁₀ is —(CH₂)ₘ—, 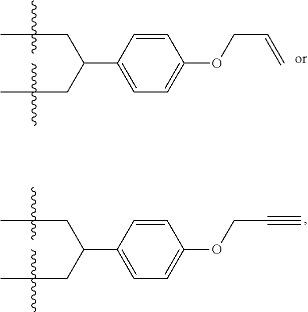 [SMILES: *CC(C*)c1ccc(OCC=C)cc1 ; *CC(C*)c1ccc(OCC#C)cc1]
m is an integer of 0-5;
R₁₁ is 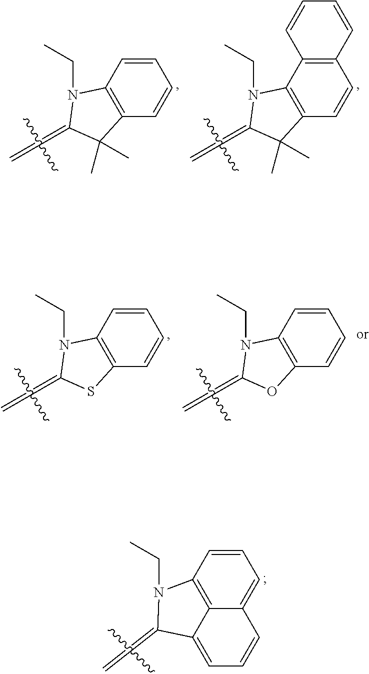 [SMILES: *=C1N(CC)c2ccccc2C1(C)C ; *=C1N(CC)c2c(ccc3ccccc23)C1(C)C ; *=C1Sc2ccccc2N1CC ; *=C1Oc2ccccc2N1CC ; *=c1c2cccc3cccc(c32)n1CC]
R₁₂ is 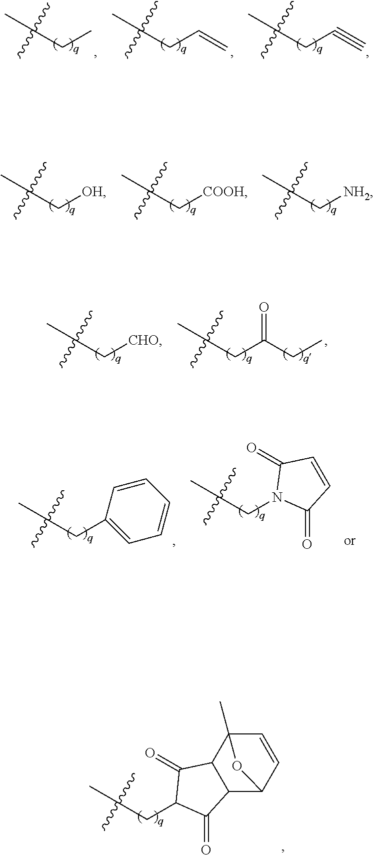
q and q′ are each independently an integer selected from 0-12.

7. A micro-nano structure wherein the micro-nano structure is formed by self-assembly of Compound II-1, II-2, II-3, II-4, II-5, II-6, II-7, II-8, II-9, II-10, II-11, II-12, II-13, II-14, II-15, II-16 or II-17 in an aqueous solution.

8. The micro-nano structure according to claim 1, wherein the micro-nano structure is a nano-sheet structure.

9. A method for preparing the micro-nano structure according to claim 1, comprising the steps of:
1) dissolving the compound, the isomer, pharmaceutically acceptable salt, hydrate or solvate thereof in an organic solvent;
2) adding the solution obtained in step 1) to an aqueous solution;
3) self-assembling the compound to form the micro-nano structure in the aqueous solution.

10. The method according to claim 9, wherein in the system formed in step 2), the compound has a final concentration of 10 nM to 1 mM.

11. A compound represented by formula (III), an isomer, a pharmaceutically acceptable salt, a hydrate or a solvate thereof, 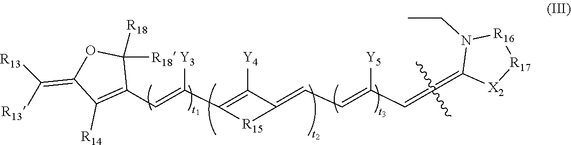
in the formula (III),
X₂ is selected from O, S or —CR₂₀R₂₀′—;
Y₃, Y₄ and Y₅ are each independently selected from H, hydroxyl, halogen atom, substituted or non-substituted amino or hydrocarbyloxy;
t₁, t₂ and t₃ are each independently an integer selected from 0-5;
R₁₃, R₁₃′ and R₁₄ are each independently selected from —CN, —CF₃, F, —SO₂CF₃, —NO₂, —COOEt, —SO₂ph, 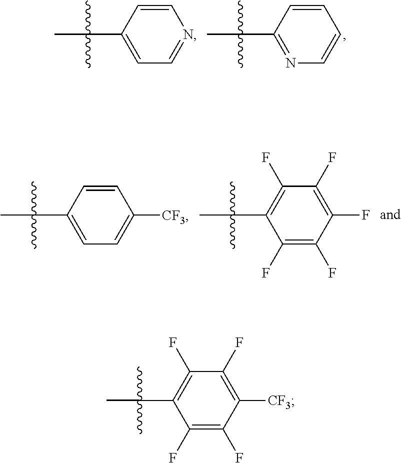 [SMILES: *c1ccncc1 ; *c1ccccn1 ; *c1ccc(C(F)(F)F)cc1 ; *c1c(F)c(F)c(F)c(F)c1F ; *c1c(F)c(F)c(C(F)(F)F)c(F)c1F]
R₁₅ is —(CH₂)ₘ—, 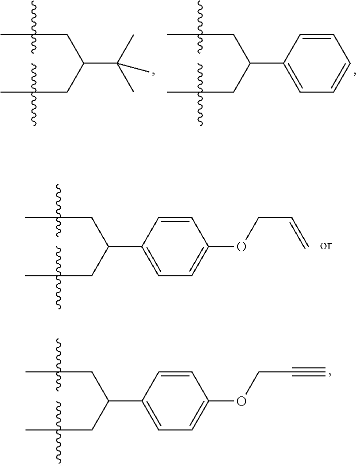
m is an integer of 0-5;
R₁₆ and R₁₇ together form one of the following connections: 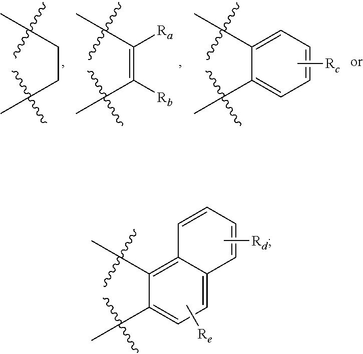
or R₁₆, R₁₇ and X₂ together form a connection 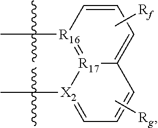
wherein Rₐ, R_{b}, R_{c}, R_{d}, Rₑ, R_{f} and R_{g} are each independently selected from H, halogen, substituted or non-substituted hydrocarbyl, substituted or non-substituted carboxyl, substituted or non-substituted hydroxyl and substituted or non-substituted amino;
R₁₈, R₁₈′, R₂₀ and R₂₀′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amido, substituted or unsubstituted ester group and substituted or unsubstituted amino;
when the group is substituted, the substituent is single or multiple.

12. A method for preparing a phototherapy drug for the diagnosis and/or treatment of cancer or for treating skin diseases, wherein the drug comprises a compound represented by formula (IV), an isomer, a pharmaceutically acceptable salt, a hydrate or a solvate thereof, 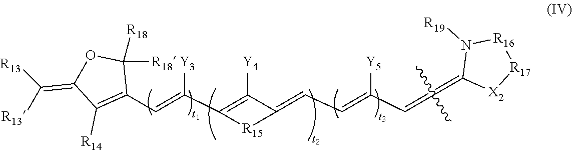
in the formula (IV),
X₂ is selected from O, S or —CR₂₀R₂₀′—;
Y₃, Y₄ and Y₅ are each independently selected from H, hydroxyl, halogen atom, substituted or unsubstituted amino and hydrocarbyloxy;
t₁, t₂ and t₃ are each independently an integer selected from 0-5;
R₁₃, R₁₃′ and R₁₄ are each independently selected from —CN, —CF₃, F, —SO₂CF₃, —NO₂, —COOEt, —SO₂ph, 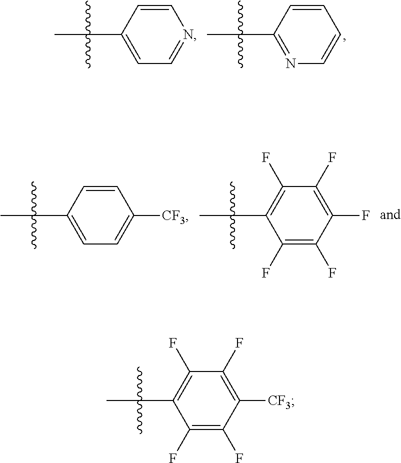 [SMILES: *c1ccncc1 ; *c1ccccn1 ; *c1ccc(C(F)(F)F)cc1 ; *c1c(F)c(F)c(F)c(F)c1F ; *c1c(F)c(F)c(C(F)(F)F)c(F)c1F]
R₁₅ is —(CH₂)ₘ—, 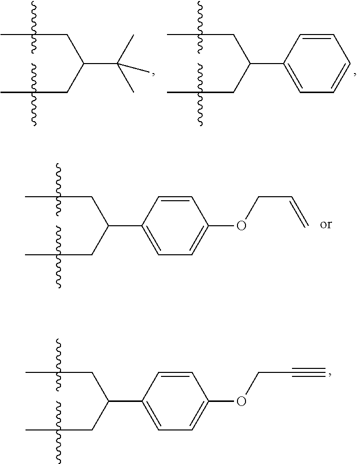
m is an integer of 0-5;
R₁₆ and R₁₇ together form one of the following connections: 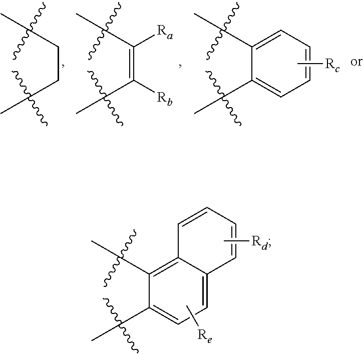
or R₁₆, R₁₇ and X₂ together form a connection 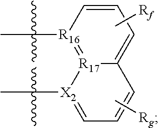
wherein Rₐ, R_{b}, R_{c}, R_{d}, Rₑ, R_{f} and R_{g} are each independently selected from H, halogen, substituted or non-substituted hydrocarbyl, substituted or non-substituted carboxyl, substituted or non-substituted hydroxyl and substituted or non-substituted amino;
R₁₈, R₁₈′, R₁₉, R₂₀ and R₂₀′ are each independently selected from H, halogen atom, substituted or non-substituted hydrocarbyl, substituted or non-substituted cyclic hydrocarbyl, substituted or non-substituted aryl, substituted or non-substituted heteroaryl, substituted or non-substituted heterocyclyl, substituted or non-substituted alcohol group, substituted or unsubstituted ether group, substituted or unsubstituted aldehyde group, substituted or unsubstituted carboxy, substituted or unsubstituted amide, substituted or unsubstituted ester group and substituted or unsubstituted amino;
when the group is substituted, the substituent is single or multiple.

13. A pharmaceutical composition comprising:
1) a therapeutically effective dose of the micro-nano structure of claim 1, and
2) pharmaceutically acceptable carrier.

14. The micro-nano structure according to claim 1, wherein the micro-nano structure is used in the preparation of a phototherapy drug, in the preparation of a drug for diagnosis and/or treatment of cancer, or in the preparation of a drug for the treatment of skin diseases.

15. The micro-nano structure according to claim 14, wherein the phototherapy drug is a photothermal therapeutic drug, a photodynamic therapeutic drug or a photoacoustic therapeutic drug, the cancer is esophageal cancer, non-small cell lung cancer, biliary cancer, head-neck cancer, Barrett esophagus, bladder cancer, colorectal cancer, pancreatic cancer, ovarian cancer, prostate cancer, brain tumor, breast cancer or skin cancer, the skin disease is actinic keratosis, basal cell carcinoma, skin T cell lymphoma, Bowen's disease, squamous cell carcinoma, intraepithelial neoplasia of the vulva and anus, or Paget's disease.
